(19) United States
(12) Patent Application Publication
(10) Pub. No.: US 2009/0163521 A1
(43) Pub. Date: Jun. 25, 2009

(54) Novel Pyrazolopyrimidinone Derivatives

(75) Inventors: Ravikumar Tadiparthi, Chennai (IN); Simi Pushpan, Chennai (IN); Sriram Rajagopal, Chennai (IN); Rajib Barik, Chennai (IN)

Correspondence Address: OLIFF & BERRIDGE, PLC, P.O. BOX 320850, ALEXANDRIA, VA, 22320-4850, US

(73) Assignee: ORCHID RESEARCH LABORATORIES LIMITED, Chennai (IN)

(21) Appl. No.: 11/921,926

(22) PCT Filed: Jun. 28, 2006
(86) PCT No.: PCT/IB2006/001791

§ 371 Date: Jan. 8, 2008

(30) Foreign Application Priority Data
Jun. 28, 2005 (IN) 813/CHE/2005

(51) Int. Cl. A61K 31/519; C07D 487/04; A61P 9/10

(52) U.S. Cl. 514/262.1; 544/262

(57) ABSTRACT

The present invention relates to novel pyrazolopyrimidinones of the general formula (I), their derivatives, their analogs, their pharmaceutically acceptable salts and pharmaceutically acceptable compositions containing them. The present invention more particularly provides novel pyrazolopyrimidinones derivatives of the general formula (I).

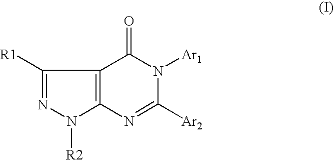

### FIELD OF THE INVENTION

The present invention relates to novel pyrazolopyrimidinones of the general formula (I), their derivatives, their analogs, their pharmaceutically acceptable salts and pharmaceutically acceptable compositions containing them. The present invention more particularly provides novel pyrazolopyrimidinones derivatives of the general formula (I).

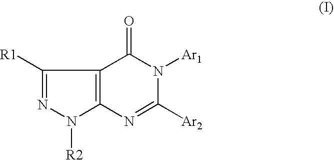

The present invention also provides a process for the preparation of the above said novel pyrazolopyrimidinones of the formula (I) pharmaceutically acceptable salts, their derivatives, their analogs, their pharmaceutically acceptable salts, and pharmaceutical compositions containing them.

The novel pyrazolopyrimidinones of the present invention are useful for the treatment of inflammation and immunological diseases. Particularly the compounds of the present invention are useful for the treatment of inflammation and immunological diseases those mediated by cytokines such as TNF-α, IL-1, IL-6, IL-1, IL-8, IL-12, MAP kinase, p38 kinase and cyclooxygenase such as COX-2 and COX-3. The compounds of the present invention are also useful for the treatment of rheumatoid arthritis; osteoporosis; multiple myeloma; uveititis; acute and chronic myelogenous leukemia; ischemic heart disease, atherosclerosis, cancer, ischemic-induced cell damage, pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; type I and type II diabetes; bone resorption diseases; ischemia reperfusion injury; atherosclerosis; brain trauma; multiple sclerosis; cerebral malaria; sepsis; septic shock; toxic shock syndrome; fever and myalgias due to infection; and as diuretic; and diseases mediated by HIV-1; HIV-2; HIV-3; cytomegalovirus (CMV); influenza; adenovirus; the herpes viruses (including HSV-1, HSV-2) and herpes zoster viruses.

### BACKGROUND OF INVENTION

The present invention is concerned with treatment of immunological diseases or inflammation, notably such diseases are mediated by cytokines or cyclooxygenase. The principal elements of the immune system are macrophages or antigen-presenting cells, T cells and B cells. The role of other immune cells such as NK cells, basophils, mast cells and dendritic cells are known, but their role in primary immunologic disorders is uncertain. Macrophages are important mediators of both inflammation and providing the necessary “help” for T cell stimulation and proliferation. Most importantly macrophages make IL-1, IL-6, IL-8, IL-12 and TNF-α all of which are potent pro-inflammatory molecules and also provide help for T cells. In addition, activation of macrophages results in the induction of enzymes, such as cyclooxygenase-2 (COX-2) and cyclooxygenase-3 (COX-3), inducible nitric oxide synthase (iNOS) and production of free radicals capable of damaging normal cells. Many factors activate macrophages, including bacterial products, superantigens and interferon gamma (IFNγ). It is believed that phosphotyrosine kinases (PTKs) and other undefined cellular kinases are involved in the activation process.

Cytokines are molecules secreted by immune cell large number of chronic and acute conditions have been recognized to be associated with perturbation of the inflammatory response. A large number of cytokines participate in this response, including IL-1, IL-6, IL-8 and TNF. It appears that the activity of these cytokines in the regulation of inflammation rely at least in part on the activation of an enzyme on the cell signaling pathway, a member of the MAP known as CSBP and RK. This kinase is activated by dual phosphorylation after stimulation by physiochemical stress, treatment with lipopolysaccharides or with proinflammatory cytokines such as IL-1 and TNF. Therefore, inhibitors of the kinase activity of p38 are useful anti-inflammatory agents.

Cytokines are molecules secreted by immune cells that are important in mediating immune responses. Cytokine production may lead to the secretion of other cytokines, altered cellular function, cell division or differentiation. Inflammation is the body's normal response to injury or infection. However, in inflammatory diseases such as rheumatoid arthritis, pathologic inflammatory processes can lead to morbidity and mortality. The cytokine tumor necrosis factor-alpha (TNF-α) plays a central role in the inflammatory response and has been targeted as a point of intervention in inflammatory disease. TNF-α is a polypeptide hormone released by activated macrophages and other cells. At low concentrations, TNF-α participates in the protective inflammatory response by activating leukocytes and promoting their migration to extravascular sites of inflammation (Moser et al., J Clin Invest, 83, 444-55, 1989). At higher concentrations, TNF-α can act as a potent pyrogen and induce the production of other pro-inflammatory cytokines (Haworth et al., Eur J Immunol, 21, 2575-79, 1991; Brennan et al., Lancet, 2, 244-7, 1989). TNF-α also stimulates the synthesis of acute-phase proteins. In rheumatoid arthritis, a chronic and progressive inflammatory disease affecting about 1% of the adult U.S. population, TNF-α mediates the cytokine cascade that leads to joint damage and destruction (Arend et al., Arthritis Rheum, 38, 151-60, 1995). Inhibitors of TNF-α, including soluble TNF receptors (etanercept) (Goldenberg, Clin Ther, 21, 75-87, 1999) and anti-TNF-α antibody (infliximab) (Luong et al., Ann Pharmacother, 34, 743-60, 2000), recently approved by the U.S. Food and Drug Administration (FDA) as agents for the treatment of rheumatoid arthritis.

Elevated levels of TNF-α have also been implicated in many other disorders and disease conditions, including cachexia, septic shock syndrome, osteoarthritis, inflammatory bowel disease such as Crohn's disease and ulcerative colitis etc.

Elevated levels of TNF-α and/or IL-1 over basal levels have been implicated in mediating or exacerbating a number of disease states including rheumatoid arthritis; osteoporosis; multiple myeloma; uveititis; acute and chronic myelogenous leukemia; pancreatic β cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; type I and type II diabetes; bone resorption diseases; ischemia reperfusion injury; atherosclerosis; brain trauma; multiple sclerosis; cerebral malaria; sepsis; septic shock; toxic shock syndrome; fever, and myalgias due to infection. HIV-1, HIV-2, HIV-3, cytomegalovirus (CMV), influenza, adenovirus, the herpes viruses (including HSV-1, HSV-2), and herpes zoster are also exacerbated by TNF-α.

It can be seen that inhibitors of TNF-α are potentially useful in the treatment of a wide variety of diseases. Compounds that inhibit TNF-α have been described in several patents.

Excessive production of IL-6 is implicated in several disease states; it is highly desirable to develop compounds that inhibit IL-6 secretion. Compounds that inhibit IL-6 have been described in U.S. Pat. Nos. 6,004,813; 5,527,546 and 5,166,137.

The cytokine IL-1β also participates in the inflammatory response. It stimulates thymocyte proliferation, fibroblast growth factor activity, and the release of prostaglandin from synovial cells. Elevated or unregulated levels of the cytokine IL-1β have been associated with a number of inflammatory diseases and other disease states, including but not limited to adult respiratory distress syndrome, allergy, Alzheimer's disease etc. Since overproduction of IL-1β is associated with numerous disease conditions, it is desirable to develop compounds that inhibit the production or activity of IL-1β.

In rheumatoid arthritis models in animals, multiple intra-articular injections of IL-1 have led to an acute and destructive form of arthritis (Chandrasekhar et al., Clinical Immunol Immunopathol. 55, 382, 1990). In studies using cultured rheumatoid synovial cells, IL-1 is a more potent inducer of stromelysin than TNF-α. (Firestein, Am. J. Pathol. 140, 1309, 1992). At sites of local injection, neutrophil, lymphocyte, and monocyte emigration has been observed. The emigration is attributed to the induction of chemokines (e.g., IL-8), and the up-regulation of adhesion molecules (Dinarello, Eur. Cytokine Netw. 5, 517-531, 1994).

In rheumatoid arthritis, both IL-1 and TNF-α induce synoviocytes and chondrocytes to produce collagenase and neutral proteases, which leads to tissue destruction within the arthritic joints. In a model of arthritis (collagen-induced arthritis (CIA) in rats and mice) intra-articular administration of TNF-α either prior to or after the induction of CIA led to an accelerated onset of arthritis and a more severe course of the disease (Brahn et al., Lymphokine Cytokine Res. 11, 253, 1992; and Cooper, Clin. Exp. Immunol. 898, 244, 1992).

IL-8 has been implicated in exacerbating and/or causing many disease states in which massive neutrophil in filtration into sites of inflammation or injury (e.g., ischemia) is mediated chemotactic nature of IL-8, including, but not limited to, the following: asthma, inflammatory bowl disease, psoriasis, adult respiratory distress syndrome, cardiac and renal reperfusion injury, thrombosis and glomerulonephritis. In addition to the chemotaxis effect on neutrophils, IL-8 has also has ability to activate neutrophils. Thus, reduction in IL-8 levels may lead to diminish neutrophil infiltration.

It has been reported that Cyclooxygenase enzyme exists in three isoforms, namely, COX-1, COX-2 and COX-3. COX-1 enzyme is essential and primarily responsible for the regulation of gastric fluids whereas COX-2 enzyme is present at the basal levels and is reported to have a major role in the prostaglandin synthesis for inflammatory response. These prostaglandins are known to cause inflammation in the body. Hence, if the synthesis of these prostaglandins is stopped by way of inhibiting COX-2 enzyme, inflammation and its related disorders can be treated. COX-3 possesses glycosylation-dependent cyclooxygenase activity. Comparison of canine COX-3 activity with murine COX-1 and COX-2 demonstrated that this enzyme is selectively inhibited by analgesic/antipyretic drugs such as acetaminophen, phenacetin, antipyrine, and dipyrone, and is potently inhibited by some nonsteroidal antiinflammatory drugs. Thus, inhibition of COX-3 could represent a primary central mechanism by which these drugs decrease pain and possibly fever. Earlier reports before to coxibs development show that inhibitors of COX-1 enzyme causes gastric ulcers, where as selective COX-2 and COX-3 enzyme inhibitors are devoid of this function and hence are found to be safe. But, recent reports show that the selective COX-2 inhibitors (COXIBs) are associated with the cardiovascular risks. So, inhibition of COX-2 without causing cardiovascular risks and gastric ulcers due to inhibition of COX-1 are showed to be safe and is concerned in the present invention.

Few prior art references, which disclose the closest compounds, are given here:

i) U.S. Pat. Nos. 5,726,124 and 5,300,477 disclose novel herbicidal compounds of formula (IIa)

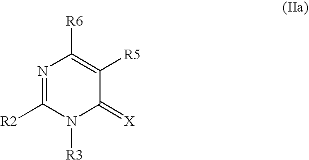

R₂ is a substituted or unsubstituted aryl group or a substituted or unsubstituted heteroaromatic group (e.g. a heteroaromatic ring structure having four to five carbon atoms and one heteroatom selected from the group consisting of nitrogen, sulfur and oxygen); R₃ is an alkyl, haloalkyl, polyhaloalkyl, haloalkenyl, polyhaloalkenyl, alkenyl, alkynyl, haloalkynyl, polyhaloalkynyl, alkoxyalkyl, dialkoxyalkyl, haloalkoxyalkyl, oxoalkyl, trimethylsilylalkynyl, cyanoalkyl or aryl group; R₅ is a hydrogen, halo, acyl, alkyl, alkenyl, alkynyl, alkoxy, alkylthio, alkoxyalkyl, alkoxyimino, alkoxycarbonylalkyl, dialkoxyalkyl, formyl, haloalkyl, haloalkenyl, haloalkynyl, haloalkoxy, hydroxyalkyl, hydroxyimino, polyhaloalkyl, polyhaloalkenyl, polyhaloalkynyl, polyhaloalkoxy, trimethylsilylalkynyl, alkoxyalkoxy, aminocarbonylalkyl, alkylaminocarbonylalkyl, dialkylaminocarbonylalkyl, cyanoalkyl, hydroxy or cyano group; and R₆ is a hydrogen, halo, alkyl, alkenyl, alkynyl, alkoxy, alkylthio, alkoxyalkyl, alkoxycarbonyl, alkoxycarbonylalkyl, haloalkyl, haloalkenyl, haloalkynyl, haloalkoxy, haloalkylthio, polyhaloalkyl, polyhaloalkenyl, polyhaloalkynyl, polyhaloalkoxy, polyhaloalkylthio, cycloalkyl, aryl, aryloxy, heterocyclyl, aralkyl, alkylamino, dialkylamino, dialkylaminocarbonyl, or cyano group; and X is oxygen or sulfur.

An example of these compounds is shown in formula (IIb)

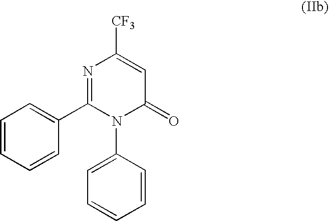

ii) U.S. Pat. No. 5,474,996 discloses novel compounds of formula (IIc)

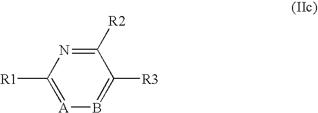

wherein

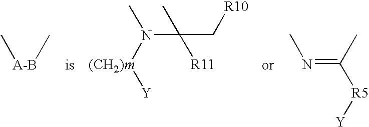

R₅ is a single bond or —(CH₂)ₘ—, —NH—, etc., m is an integer of 0 to 4; Y is Y₁—B—Y₂ is a monocyclic aryl of 5 to 6 ring member or condensed ring of 8 to 10 ring members optionally containing at least one heteroatom chosen from oxygen, nitrogen and sulfur; R₁₀ and R₁₁ together form oxo group; R₂ is chosen from the group consisting of hydrogen, halogen, hydroxyl, mercapto, cyano, nitro, formyl, benzoyl, acyl of 1 to 6 carbon atoms, alkyl, alkenyl, alkoxy, alkylthio of up to 10 carbon atoms, phenyl, phenoxy, naphthyl, benzyl, phenylthio, biphenyl, biphenylmethyl and indole; R₃ is alkyl substituted with carboxy or esterified carboxy.

An example of these compounds is shown in formula (IId)

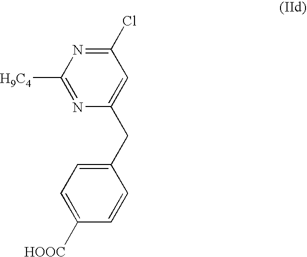

iii) U.S. Pat. Nos. 6,420,385 and 6,410,729 discloses novel compounds of formula (IIe)

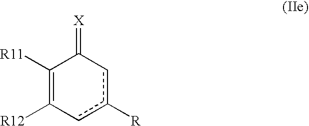

wherein

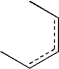

represents

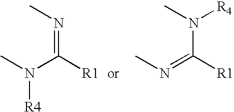

X is O, S or NR₅; R₁ and R₂ are each independently represent —Y or -Z-Y, and R₃ and R₄ are each independently -Z-Y or R₃ is a hydrogen radical; provided that R₄ is other than a substituted-aryl, (substituted-aryl)methyl or (substituted-aryl)ethyl radical; wherein each Z is independently optionally substituted alkyl, alkenyl, alkynyl, heterocyclyl, aryl or heteroaryl; Y is independently a hydrogen; halo, cyano, nitro, etc., R₅ is independently a hydrogen, optionally substituted alkyl, alkenyl, alkynyl etc., R₁₁ and R₁₂ each independently represent optionally substituted aryl or heteroaryl.

An example of these compounds is shown in formula (IIf)

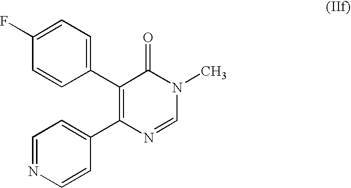

iv) U.S. Pat. No. 4,771,040 discloses 6-oxopyrimidinyl(thiono)phosphate pesticide compounds and intermediate of formula (IIg)

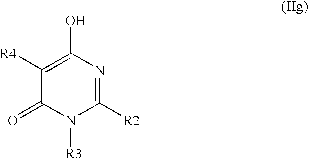

wherein R₂ represents hydrogen, optionally substituted alkyl, or alkoxy, alkylthio, dialkylamino or aryl; R₃ represents alkyl or aryl; Rx represents hydrogen, halogen or alkyl.

An example of these compounds is shown in formula (IIh)

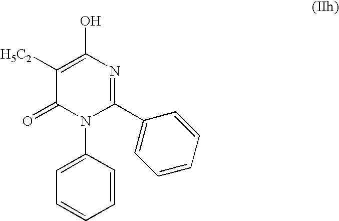

v) DE 2142317 discloses hypnotic uracil derivatives of formula (Hi)

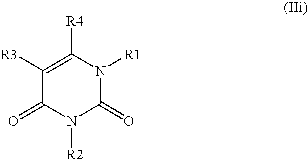

wherein R₁ is H, alkyl, alkenyl, dialkylaminoalkyl, or aralkyl; R₂ is H, alkyl, aryl, or halogen; R₃ is alkyl, alkenyl, cycloalkyl, aralkyl, aralkenyl, or aryl, R₄ is alkyl, alkenyl, cycloalkyl, aralkyl, aryl, etc.

An example of these compounds is shown in formula (IIj)

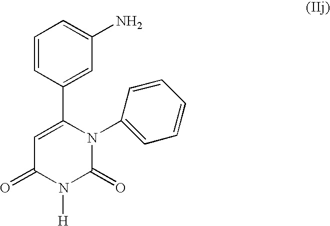

vi) U.S. Pat. No. 5,470,975 discloses dihydropyrimidine derivatives of formula (IIk)

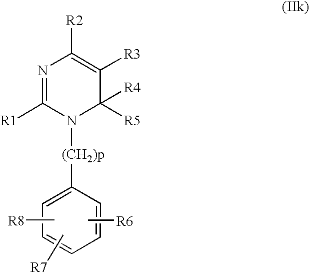

R₁ is alkyl, alkenyl, alkynyl, cycloalkyl, NR₄R₅ etc., R₂ is hydrogen, halogen, SR₄, etc., R₃ is R₄, —COOR, —CONH₂, CN, etc., R₄, R₅ are independently selected from hydrogen, alkyl, alkenyl, alkynyl, aryl, arylalkyl, cycloalkyl etc., or R₄ and R₅ together with the carbon atoms to which they are attached form a carbonyl or a thiocarbonyl group; R₆ is —CN, alkyl, acyloxy, SO₂NH₂, aryl, furyl; R₇ is H, halogen, etc., R₈ is H, halogen, alkyl, alkoxy etc.,

An example of these compounds is shown in formula (III)

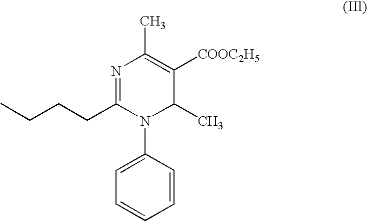

(vii) EP 1460077 A1 The present invention relates to novel pyrazolopyrimidones, compositions comprising pyrazolopyrimidones as well as to the use of compounds and the composition for the production of a medicament acting as a PDE inhibitor, such as for the treatment of erectile dysfunction. Compound, represented by one of the structural formulas:

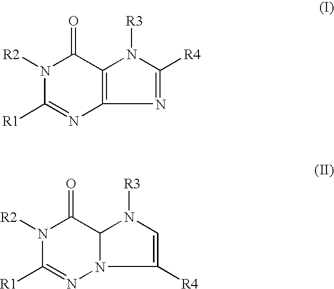

or mixtures thereof wherein R₁, R₂, R₃, and R₄ are independently hydrogen, halogen, hydroxyl, amino, alkyl, alkenyl, alkynyl, cycloalkyl, cycloalkenyl, cycloalkynyl, haloalkyl, alkylaryl, aryl, aralkyl, alkoxy, carboxy or heterocyclyl, all of these substituents being substituted or unsubstituted, with the exception of formula (XI), wherein R₁ being hydrogen, C₁-C₄ alkyl, C₁-C₄ haloalkyl, piperidinomethyl, methoxymethyl, N-methylpiperazino methyl, carbethoxy, p-chlorophenoxymethyl or Ar—(CH₂)ₙ—, wherein n is 0-4;

### OBJECTIVE OF THE INVENTION

We have focused our research to identify cytokine inhibitors with predominantly acting through the inhibition of tumour necrosis factor-α (TNF-α) which are devoid of any side effects normally associated with tumour necrosis factor-α TNF-α) inhibitors. Our sustained efforts have resulted in novel compounds of the formula (I). The derivatives may be useful in the treatment of inflammation and immunological diseases. Particularly the compounds of the present invention are useful for the treatment of immunological diseases those mediated by cytokines such as TNF-α, IL-1, IL-6, IL-1β, IL-8, IL-12 and inflammation. The compounds of the present invention are also useful in the treatment of rheumatoid arthritis; osteoporosis; multiple myeloma; uveititis; acute and chronic myelogenous leukemia; ischemic heart disease; atherosclerosis; cancer; ischemic-induced cell damage; pancreatic β-cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; asthma; muscle degeneration; cachexia; bone resorption diseases; ischemia reperfusion injury; atherosclerosis; brain trauma; multiple sclerosis; sepsis; septic shock; toxic shock syndrome; fever, and myalgias due to infection.

### SUMMARY OF THE INVENTION

The present invention relates to novel pyrazolopyrimidinone derivatives of the formula (I)

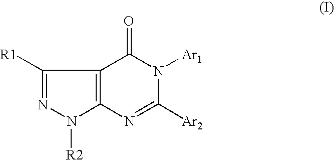

their derivatives, their pharmaceutically acceptable salts and their pharmaceutically acceptable compositions, wherein Ar₁ and Ar₂ may be same or different and independently represent substituted or unsubstituted groups selected from aryl, heteroaryl, heterocyclyl group; R₁ represents hydrogen, hydroxyl, halogen, formyl, amino, hydrazine, alkylamino, arylamino, acylamino, sulfonylamino, substituted (C₁-C₆)alkyl, substituted or unsubstituted groups selected from acyl, aryl, aralkyl, heteroaryl, heteroaralkyl, heterocyclyl, —NHCH₂CN, —NHCH₂C(═NH)NHOH, NHCONH₂, —NHCSNH₂, —NHCONH-alkyl, —NHCONH-aryl, —NHCSNH-alkyl, —NHCSNH-aryl, —NHCO-aryl, —NHCO-heteroaryl, NHCO-piperzine, —NHCS-piperzine; R₂ represents hydrogen, hydroxy, nitro, nitroso, alkyl, azido, halogen, —C(—NH)NH₂, formyl, substituted or unsubstituted groups selected from haloalkyl, alkoxy, aryloxy, aralkyl, aralkoxy, heteroaryl, heterocyclyl, acyl, acyloxy, cycloalkyl, amino, monoalkylamino, dialkylamino, acylamino, alkylsulfonyl, arylsulfonyl, alkylsulfinyl, arylsulfinyl, alkylthio, arylthio, alkoxycarbonyl, aryloxycarbonyl, alkoxyalkyl, sulfamoyl, carboxylic acid and its derivatives.

### DETAILED DESCRIPTION OF THE INVENTION

Suitable groups represented by Ar₁ and Ar₂ are selected from aryl group such as phenyl or naphthyl, the aryl group may be substituted; heteroaryl group may be mono or fused system such as pyridyl, thienyl, furyl, pyrrolyl, oxazolyl, thiazolyl, imidazolyl, oxadiazolyl, thiadiazolyl, tetrazolyl, pyrimidinyl, pyrazine, piperazine, benzopyranyl, benzofuranyl, benzimidazolyl, benzoxazolyl, benzothiazolyl, benzopyrrolyl, benzoxadiazolyl, benzothiadiazolyl and the like, the heteroaryl group may be substituted; heterocyclyl group such as pyrrolidinyl, thiazolidinyl, oxazolidinyl, morpholinyl, thiomorpholinyl, piperidinyl, piperazinyl, and the like, the heterocyclyl group may be substituted.

The substituents on the groups represented by Ar₁ and Ar₂ are selected from hydroxy, nitro, nitroso, formyl, azido, halo or substituted or unsubstituted groups selected from alkyl, haloalkyl, alkoxy, aryl, aryloxy, aralkyl, aralkoxy, heteroaryl, heterocyclyl, acyl, acyloxy, cycloalkyl, amino, hydrazine, monoalkylamino, dialkylamino, acylamino, alkylsulfonyl, arylsulfonyl, alkylsulfinyl, arylsulfinyl, alkylthio, arylthio, alkoxycarbonyl, aryloxycarbonyl, alkoxyalkyl, sulfamoyl, —SO₂N₃, —SO₂NHNH₂, —SO₂NHR₃, —SO₂NHCOR₃, —SO₂NHNHCOR₃, R₃ may be alkyl, haloalkyl, aryl, heteroaryl, -carboxylic acid and its derivatives;

Suitable groups represented by R₁ are selected from hydrogen, hydroxyl, amino, alkylamino, arylamino, acylamino, sulfonylamino, hydrazine, halogen atom such as fluorine, chlorine, bromine or iodine; formyl, substituted or unsubstituted linear or branched (C₁-C₆) alkyl group, such as methyl, ethyl, n-propyl, isopropyl, n-butyl, isobutyl, t-butyl, n-pentyl, isopentyl, hexyl and the like; acyl group such as —C(═O)CH₃, —C(═O)C₂H₅, —C(═O)C₃H₇, —C(═O)C₆H₁₃, —C(═S)CH₃, —C(═S)C₂H₅, —C(═S)C₃H₇, —C(═S)C₆H₁₃, benzoyl and the like, which may be substituted; —NHCH₂CN, —NHCH₂C(═NH)NHOH, NHCONH₂, —NHCSNH₂, —NHCONH-alkyl, —NHCONH-aryl, —NHCSNH-alkyl, —NHCSNH-aryl, —NHCO-aryl, —NHCO-heteroaryl, —NHCO-piperzine, —NHCS-piperzine; aryl group such as phenyl or naphthyl, the aryl group may be substituted; aralkyl group such as benzyl, phenylethyl, phenyl propyl and the like, which may be substituted; heteroaryl group may be mono or fused system such as pyridyl, thienyl, furyl, pyrrolyl, oxazolyl, thiazolyl, imidazolyl, oxadiazolyl, thiadiazolyl, tetrazolyl, pyrimidinyl, pyrazine, piperazine, benzopyranyl, benzofuranyl, benzimidazolyl, benzoxazolyl, benzothiazolyl, benzopyrrolyl, benzoxadiazolyl, benzothiadiazolyl and the like, the heteroaryl group may be substituted; heterocyclyl group such as pyrrolidinyl, thiazolidinyl, oxazolidinyl, morpholinyl, thiomorpholinyl, piperidinyl, piperazinyl, and the like, the heterocyclyl group may be substituted; heteroaralkyl wherein the heteroaryl group is as defined above.

Suitable groups represented by R₂ are selected from hydrogen, hydroxy, nitro, nitroso, formyl, azido, —C(═NH)NH₂, halogen atom such as fluorine, chlorine, bromine or iodine; or substituted or unsubstituted linear or branched (C₁-C₆) alkyl group, such as methyl, ethyl, n-propyl, isopropyl, n-butyl, isobutyl, t-butyl, n-pentyl, isopentyl, hexyl and the like; haloalkyl such as chloromethyl, chloroethyl, trifluoromethyl, trifluoroethyl, dichloromethyl, dichloroethyl and the like, which may be substituted; aryl group such as phenyl or naphthyl, the aryl group may be substituted; cyclo (C₃-C₆) alkyl group such as cyclopropyl, cyclobutyl, cyclopentyl, cyclohexyl and the like, the cycloalkyl group may be substituted; acyl group such as —C(═O)CH₃, —C(═O)C₂H₅, —C(═O)C₃H₇, —C(═O)C₆H₁₃, —C(═S)CH₃, —C(═S)C₂H₅, —C(═S)C₃H₇, —C(—S)C₆H₁₃, benzoyl and the like, which may be substituted; linear or branched (C₁-C₆) alkoxy group, such as methoxy, ethoxy, n-propoxy, isopropoxy and the like; aryloxy group such as phenoxy, napthoxy, the aryloxy group may be substituted; aralkoxy group such as benzyloxy, phenethyloxy and the like, which may be substituted; acyloxy group such as MeCOO—, EtCOO—, PhCOO— and the like, which may be substituted; heterocyclyl groups such as pyrrolidinyl, morpholinyl, thiomorpholinyl, piperidinyl, piperazinyl, and the like, the heterocyclyl group may be substituted; heteroaryl group may be mono or fused system such as pyridyl, thienyl, furyl, pyrrolyl, oxazolyl, thiazolyl, imidazolyl, oxadiazolyl, thiadiazolyl, tetrazolyl, pyrimidinyl, pyrazine, piperazine, benzopyranyl, benzofuranyl, benzimidazolyl, benzoxazolyl, benzothiazolyl, benzopyrrolyl, benzoxadiazolyl, benzothiadiazolyl and the like, the heteroaryl group may be substituted; aralkyl group such as benzyl, phenylethyl, phenyl propyl and the like, which may be substituted; amino, which may be substituted; hydrazine, which may be substituted; monoalkylamino group such as —NHCH₃, —NHC₂H₅, —NHC₃H₇, —NHC₆H₁₃, and the like, which may be substituted; dialkylamino group such as —N(CH₃)₂, —NCH₃(C₂H₅), —N(C₂H₅)₂ and the like, which may be substituted; acylamino group such as —NHC(═O)CH₃, —NHC(═O)C₂H₅, —NHC(═O)C₃H₇, —NHC(═O)C₆H₁₃, and the like, which may be substituted; alkoxycarbonyl group such as methoxycarbonyl, ethoxycarbonyl, n-propoxycarbonyl, isopropoxycarbonyl and the like, the alkoxycarbonyl group may be substituted; aryloxycarbonyl group such as phenoxycarbonyl, napthoxycarbonyl, the aryloxycarbonyl group may be substituted; alkylsulfonyl group such as methylsulfonyl, ethylsulfonyl, n-propylsulfonyl, iso-propylsulfonyl and the like, the alkylsulfonyl group may be substituted; arylsulfonyl group such as phenylsulfonyl or naphthylsulfonyl, the arylsulfonyl group may be substituted; alkylsulfinyl group such as methylsulfinyl, ethylsulfinyl, n-propylsulfinyl, iso-propylsulfinyl and the like, the alkylsulfinyl group may be substituted; arylsulfinyl group such as phenylsulfinyl or naphthylsulfinyl, the arylsulfinyl group may be substituted; alkylthio group such as methylthio, ethylthio, n-propylthio, iso-propylthio and the like, the alkylthio group may be substituted; arylthio group such as phenylthio, or naphthylthio, the arylthio group may be substituted; alkoxyalkyl group such as methoxymethyl, ethoxymethyl, methoxyethyl, ethoxyethyl and the like, which may be substituted; sulfamoyl; carboxylic acid and its derivatives such as esters, amides and acid halides.

When the groups R₁, R₂, are substituted, the substituents are selected from halogen, hydroxy, nitro, cyano, azido, nitroso, amino, hydrazine, formyl, alkyl, aryl, cycloalkyl, alkoxy, aryloxy, acyl, acyloxyacyl, heterocyclyl, heteroaryl, monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups or carboxylic acids and its derivatives and these substituents are as defined above.

Representative Compounds According to the Present Invention Include:
3-Amino-5-(4-methylphenyl)-6-[4-(methylthio)phenyl]-1,5-dihydro-4H-pyrazolo[3,4-d]pyrimidin-4-one;
3-Amino-5-(3,4-dimethylphenyl)-6-[4-(methylthio)phenyl]-1,5-dihydro-4H-pyrazolo[3,4-d]pyrimidin-4-one;
3-Amino-5-(4-fluorophenyl)-6-[4-(methylthio)phenyl]-1,5-dihydro-4H-pyrazolo[3,4-d]pyrimidin-4-one;
3-Amino-5-[(4-methylthio)phenyl]-6-(4-fluorophenyl)-1,5-dihydro-4H-pyrazolo[3,4-d]pyrimidin-4-one;
3-Amino-5-(4-tert-butylphenyl)-6-[4-(methylthio)phenyl]-1,5-dihydro-4H-pyrazolo[3,4-d]pyrimidin-4-one;
3-Amino-5-(4-isopropylphenyl)-6-[(4-methylthio)phenyl]-1,5-dihydro-4H-pyrazolo[3,4-d]pyrimidin-4-one;
3-Amino-5-[(4-methylthio)phenyl]-6-(4-chlorophenyl)-1,5-dihydro-4H-pyrazolo[3,4-d]pyrimidin-4-one;
3-Amino-5-(4-chlorophenyl)-6-[(4-methylthio)phenyl]-1,5-dihydro-4H-pyrazolo[3,4-d]pyrimidin-4-one;
3-Amino-5-(4-ethylphenyl)-6-[(4-methylthio)phenyl]-1,5-dihydro-4H-pyrazolo[3,4-d]pyrimidin-4-one;
3-Amino-5-[(4-methylthio)phenyl]-6-phenyl-1,5-dihydro-4H-pyrazolo[3,4-d]pyrimidin-4-one;
3-Amino-5-(4-fluorophenyl)-6-phenyl-1,5-dihydro-4H-pyrazolo[3,4-d]pyrimidin-4-one;
3-Amino-5-[(4-methylthio)phenyl]-6-(4-methylphenyl)-1,5-dihydro-4H-pyrazolo[3,4-d]pyrimidin-4-one;
3-Amino-5-(4-methylphenyl)-6-[(4-methylsulfonyl)phenyl]-1,5-dihydro-4H-pyrazolo[3,4-d]pyrimidin-4-one;
3-Amino-5-(4-ethoxyphenyl)-6-[(4-methylthio)phenyl]-1,5-dihydro-4H-pyrazolo[3,4-d]pyrimidin-4-one;
3-Amino-5-(4-methylphenyl)-6-phenyl-1,5-dihydro-4H-pyrazolo[3,4-d]pyrimidin-4-one;
3-Amino-5-[(4-methylthio)phenyl]-6-(4-trifluoromethylphenyl)-1,5-dihydro-4H-pyrazolo[3,4-d]pyrimidin-4-one;
4-[3-Amino-5-(4-methylphenyl)-4-oxo-4,5-dihydro-1H-pyrazolo[3,4-d]pyrimidin-6-yl]benzenesulfonamide;
3-Amino-5-(4-bromophenyl)-6-[(4-methylthio)phenyl]-1,5-dihydro-4H-pyrazolo[3,4-d]pyrimidin-4-one;
3-Amino-5-(3,4-dimethylphenyl)-1-methyl-6-[4-(methylthio)phenyl]-1,5-dihydro-4H-pyrazolo[3,4-d]pyrimidin-4-one;
3-Amino-5-(4-ethoxyphenyl)-1-methyl-6-[4-(methylthio)phenyl]-1,5-dihydro-4H-pyrazolo[3,4-d]pyrimidin-4-one;
3-Amino-5-(4-isopropylphenyl)-1-methyl-6-[4-(methylthio)phenyl]-1,5-dihydro-4H-pyrazolo[3,4-d]pyrimidin-4-one;
3-Amino-5-(2,4-dimethylphenyl)-1-methyl-6-[4-(methylthio)phenyl]-1,5-dihydro-4H-pyrazolo[3,4-d]pyrimidin-4-one;
3-Amino-5-(4-chlorophenyl)-1-methyl-6-[4-(methylthio)phenyl]-1,5-dihydro-4H-pyrazolo[3,4-d]pyrimidin-4-one;
3-Amino-5-(4-fluorophenyl)-1-methyl-6-[4-(methylthio)phenyl]-1,5-dihydro-4H-pyrazolo[3,4-d]pyrimidin-4-one;
3-Amino-5-(4-methoxyphenyl)-1-methyl-6-[4-(methylthio)phenyl]-1,5-dihydro-4H-pyrazolo[3,4-d]pyrimidin-4-one;
3-Amino-5-(4-methoxyphenyl)-1-methyl-6-pyridin-4-yl-1,5-dihydro-4H-pyrazolo[3,4-d]pyrimidin-4-one;
3-Amino-5-(4-ethoxyphenyl)-1-methyl-6-pyridin-3-yl-1,5-dihydro-4H-pyrazolo[3,4-d]pyrimidin-4-one;
3-Amino-5-(4-methoxyphenyl)-1-methyl-6-pyridin-3-yl-1,5-dihydro-4H-pyrazolo[3,4-d]pyrimidin-4-one;
3-Amino-5-(4-bromophenyl)-1-methyl-6-[4-(methylthio)phenyl]-1,5-dihydro-4H-pyrazolo[3,4-d]pyrimidin-4-one;
3-Amino-6-[4-(dimethylamino)phenyl]-5-(4-methoxyphenyl)-1-methyl-1,5-dihydro-4H-pyrazolo[3,4-d]pyrimidin-4-one;
3-Amino-5-(2,4-dimethylphenyl)-1-(2-hydroxyethyl)-6-[4-(methylthio)phenyl]-1,5-dihydro-4H-pyrazolo[3,4-d]pyrimidin-4-one;
3-Amino-1-(2-hydroxyethyl)-5-(4-methoxyphenyl)-6-pyridin-3-yl-1,5-dihydro-4H-pyrazolo[3,4-d]pyrimidin-4-one;
3-Amino-6-[4-(dimethylamino)phenyl]-1-methyl-5-[(4-methylthio)phenyl]-1,5-dihydro-4H-pyrazolo[3,4-d]pyrimidin-4-one;
N-[5-(3,4-Dimethylphenyl)-4-oxo-6-[(4-methylthio)phenyl]-4,5-dihydro-1H-pyrazolo[3,4-d]pyrimidin-3-yl]acetamide;
1-Acetyl-3-amino-5-(3,4-dimethylphenyl)-6-[(4-methylthio)phenyl]-1,5-dihydro-4H-pyrazolo[3,4-d]pyrimidin-4-one;
3-Amino-5-(4-methoxyphenyl)-6-[4-(methylthio)phenyl]-1,5-dihydro-4H-pyrazolo[3,4-d]pyrimidin-4-one;
N-(4-Oxo-5-(2,4-dimethylphenyl)-1-methyl-6-[(4-methylthio)phenyl]-4,5-dihydro-1H-pyrazolo[3,4-d]pyrimidin-3-yl)-4-(trifluoromethyl)benzamide;
N-{5-(2,4-Dimethylphenyl)-1-methyl-6-[4-(methylthio)phenyl]-4-oxo-4,5-dihydro-1H-pyrazolo[3,4-d]pyrimidin-3-yl}acetamide;
2,2,2-trifluoro-N-{5-(2,4-dimethylphenyl)-1-methyl-6-[4-(methylthio)phenyl]-4-oxo-4,5-dihydro-1H-pyrazolo[3,4-d]pyrimidin-3-yl}acetamide;
2,2,2-trifluoro-N-[5-(4-methoxyphenyl)-1-methyl-4-oxo-6-pyridin-3-yl-4,5-dihydro-1H-pyrazolo[3,4-d]pyrimidin-3-yl]acetamide;
N-(4-Oxo-5-(4-chlorophenyl)-1-methyl-6-[(4-methylthio)phenyl]-4,5-dihydro-1H-pyrazolo[3,4-d]pyrimidin-3-yl)-3-fluorobenzamide;
3-Amino-5-(4-methoxyphenyl)-6-[4-methylphenyl]-1,5-dihydro-4H-pyrazolo[3,4-d]pyrimidin-4-one;
3-Amino-5-(4-methoxyphenyl)-6-[4-methoxyphenyl]-1,5-dihydro-4H-pyrazolo[3,4-d]pyrimidin-4-one;
3-Amino-5-(4-methoxyphenyl)-6-[4-chlorophenyl]-1,5-dihydro-4H-pyrazolo[3,4-d]pyrimidin-4-one;
3-Amino-5-(4-chlorophenyl)-6-[4-chlorophenyl]-1,5-dihydro-4H-pyrazolo[3,4-d]pyrimidin-4-one;
3-Amino-5-(4-chlorophenyl)-6-[4-methoxyphenyl]-1,5-dihydro-4H-pyrazolo[3,4-d]pyrimidin-4-one;
3-Amino-5-(4-chlorophenyl)-6-[4-methylphenyl]-1,5-dihydro-4H-pyrazolo[3,4-d]pyrimidin-4-one;
3-Amino-5-(4-fluorophenyl)-6-[4-chlorophenyl]-1,5-dihydro-4H-pyrazolo[3,4-d]pyrimidin-4-one;
3-Amino-5-(4-fluorophenyl)-6-[4-methoxyphenyl]-1,5-dihydro-4H-pyrazolo[3,4-d]pyrimidin-4-one;
3-Amino-5-(4-fluorophenyl)-6-[4-methylphenyl]-1,5-dihydro-4H-pyrazolo[3,4-d]pyrimidin-4-one;
3-Amino-5-(4-trifluoromethylphenyl)-6-[4-chlorophenyl]-1,5-dihydro-4H-pyrazolo[3,4-d]pyrimidin-4-one;
3-Amino-5-(4-trifluoromethylphenyl)-6-[4-methoxyphenyl]-1,5-dihydro-4H-pyrazolo[3,4-d]pyrimidin-4-one;
3-Amino-5-(4-trifluoromethylphenyl)-6-[4-methylphenyl]-1,5-dihydro-4H-pyrazolo[3,4-d]pyrimidin-4-one;
3-Amino-5-(4-trifluoromethylphenyl)-6-[4-(methylthio)phenyl]-1,5-dihydro-4H-pyrazolo[3,4-d]pyrimidin-4-one;
3-Amino-5-(4-methylsulphonyl)-6-[4-chlorophenyl]-1,5-dihydro-4H-pyrazolo[3,4-d]pyrimidin-4-one;
3-Amino-5-(4-methylsulphonyl)-6-[4-methoxyphenyl]-1,5-dihydro-4H-pyrazolo[3,4-d]pyrimidin-4-one;
3-Amino-5-(4-methylsulphonyl)-6-[4-methylphenyl]-1,5-dihydro-4H-pyrazolo[3,4-d]pyrimidin-4-one;
3-Amino-5-(4-methylsulphonyl)-6-[4-(methylthio)phenyl]-1,5-dihydro-4H-pyrazolo[3,4-d]pyrimidin-4-one;
3-Amino-5-[4-(methylthio)phenyl]-6-pyridin-4-yl-1,5-dihydro-4H-pyrazolo[3,4-d]pyrimidin-4-one;
3-Amino-5-(4-ethoxyphenyl)-1-methyl-6-pyridin-4-yl-1,5-dihydro-4H-pyrazolo[3,4-d]pyrimidin-4-one;
1-{1-Methyl-5-[4-(methylthio)phenyl]-4-oxo-6-phenyl-4,5-dihydro-1H-pyrazolo[3,4-d]pyrimidin-3-yl}urea and
1-{5-[4-(Methylthio)phenyl]-4-oxo-6-phenyl-4,5-dihydro-1H-pyrazolo[3,4-d]pyrimidin-3-yl}urea.

According to another embodiment of the present invention, there is provided a process for the preparation of novel pyrazolo[3,4-d]pyrimidin-4-one derivatives of the formula (I) wherein R₁ represents amino and all other symbols are as defined earlier as shown in scheme I given below:

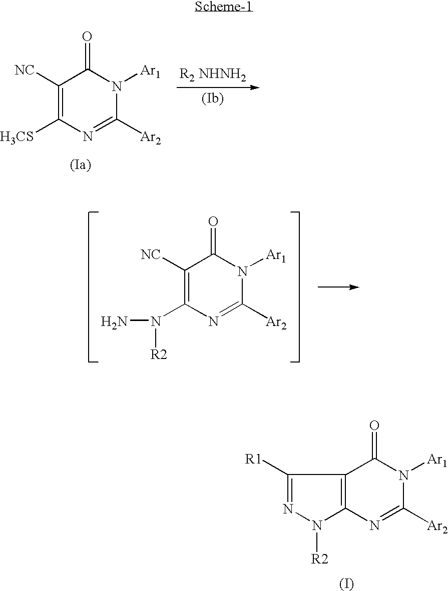

The reaction of compound of formula (Ia) with compound of formula (Ib) may be carried out using solvents like toluene, xylene, tetrahydrofuran, dioxane, chloroform, dichloromethane, dichloroethane, o-dichlorobenzene, acetone, ethylacetate, acetonitrile, N,N-dimethylformamide, dimethylsulfoxide, ethanol, methanol, isopropylalcohol, tert-butylalcohol, acetic acid, propionic acid etc., a mixture thereof or the like in the presence of base such as carbonates, bicarbonates, hydrides, hydroxides, alkoxides of alkali metals and alkaline earth metals or by neat reaction. The reaction may be carried out at a temperature in the range of 20° C. to 150° C. for period in the range of 1 to 12 h.

It is appreciated that in any of the above-mentioned reactions, any reactive group in the substrate molecule may be protected according to conventional chemical practice. Suitable protecting groups in any of the above-mentioned reactions are those used conventionally in the art. The methods of formation and removal of such protecting groups are those conventional methods appropriate to the molecule being protected.

The pharmaceutical composition may be in the forms normally employed, such as tablets, capsules, powders, syrups, solutions, aerosols, suspensions and the like, may contain flavoring agents, sweeteners etc. in suitable solid or liquid carriers or diluents, or in suitable sterile media to form injectable solutions or suspensions. Such compositions typically contain from 1 to 20%, preferably 1 to 10% by weight of the active compound, the remainder of the composition being the pharmaceutically acceptable carriers, diluents or solvents.

The present invention is provided by the examples given below, which are provided by way of illustration only and should not be considered to limit the scope of the invention.

### Example 1

### Synthesis of 3-amino-5-(4-methylphenyl)-6-[4-(methylthio)phenyl]-1,5-dihydro-4H-pyrazolo[3,4-d]pyrimidin-4-one

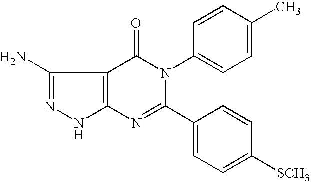

Hydrazine hydrate (0.658 g, 13 mmol) was added to a suspension of 5-cyano-1-(4-methylphenyl)-4-methylthio-2-(4-methylthiophenyl)-1,6-dihydro-pyrimidin-6-one (5.0 g, 13 mmol) (prepared according to the procedure disclosed in our PCT publication No. 03/84938) in toluene (70 ml) under stirring at room temperature. Anhydrous potassium carbonate (0.1 g, 0.7 mmol) was added to the reaction mass and heated to 70° C. for 3 h. The solid separated was filtered, washed with toluene, water and dried to yield the title compound (2.08 g, 43.5%, mp>285° C., purity 98.4% by HPLC). ¹H-NMR (DMSO-d₆): δ 2.25 (s, 3H), 2.41 (s, 3H), 5.31 (bs, 2H, D₂O exchangeable), 7.04-7.09 (m, 6H), 7.23-7.25 (d, 2H), 12.5 (bs, 1H, D₂O exchangeable). IR (KBr) cm⁻¹: 3379, 3296, 3164, 1701. MS m/z: 364 (M⁺+1).

### Example 2

### Synthesis of 3-amino-5-(3,4-dimethylphenyl)-6-[4-(methylthio)phenyl]-1,5-dihydro-4H-pyrazolo[3,4-d]pyrimidin-4-one

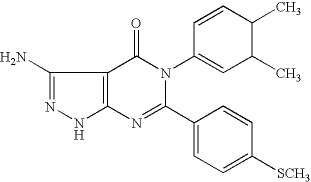

Hydrazine hydrate (1.02 g, 20.3 mmol) was added to a suspension of 5-cyano-1-(3,4-dimethylphenyl)-4-methylthio-2-(4-methylthiophenyl)-1,6-dihydro-pyrimidin-6-one (prepared according to the procedure disclosed in our PCT publication No. 03/84938) (4.0 g, 10.1 mmol) in toluene (70 ml) under stirring at room temperature. Anhydrous potassium carbonate (0.1 g, 0.7 mmol) was added to the reaction mass and heated to 70° C. for 3 hours. The solid separated was filtered, washed with water and dried. The crude product thus obtained was purified by column chromatography (silica gel 60-120 mesh) using ethylacetate and hexane mixture as eluent to yield the title compound (1.0 g, 26.3%, mp 263-266° C., purity 98.6% by HPLC). ¹H-NMR (CDCl₃): δ 1.75 (bs, 2H, D₂O exchangeable), (2.15-2.19 (d, 6H), 2.41 (s, 3H), 5.5 (bs, 1H, D₂O exchangeable), 6.89-6.9 (d, 1H), 6.99-7.0 (d, 1H), 7.01-7.02 (m, 3H), 7.20-7.22 (d, 2H). IR (KBr) cm⁻¹: 3306, 3193, 2918, 1693. MS m/z: 378.3 (M⁺+1).

### Example 3

### General Procedure

The equimolar or more than eq. molar quantities of the 1,2-diaryl-5-cyano-4-methylthio-1,6-dihydro-pyrimidin-6-one (prepared according to the procedure disclosed in our PCT publication No. 03/84938) and hydrazine hydrate or substituted or unsubstituted alkyl hydrazine were heated at 60-70° C. in toluene in the presence of half molar quantity of anhydrous potassium carbonate for 34 hrs. The resulted solid separated was filtered, washed with toluene, water and dried to yield the title compound. Purified by the column chromatography or by recrystallization techniques.

### The Following Compounds Listed in Table-I are Prepared by the General Procedure Given in Example 3

TABLE I

| EXP. | STRUCTURE | ANALYTICAL DATA |
|---|---|---|
| 4 | 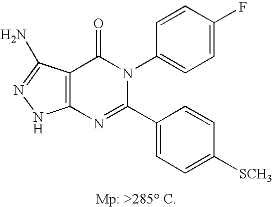 | Purity (HPLC): 99.85%, ¹H-NMR (DMSO-d₆): δ 2.41 (s, 3H), 5.2 (s, 2H, D₂O exchangeable), 7.05-7.14 (m, 4H), 7.21-7.28 (m, 4H), 12.5 (s, 1H, D₂O exchangeable). IR (KBr) cm⁻¹: 3451, 3292, 3131, 2918, 1694. MS m/z: 368.1 (M⁺ + 1). |
| 5 | 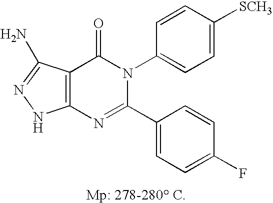 | Purity (HPLC): 96.58%, ¹H-NMR (DMSO-d₆): δ 2.42 (s, 3H), 5.3 (s, 2H, D₂O exchangeable), 7.05-7.15 (m, 6H), 7.37-7.40 (m, 2H), 12.5 (s, 1H, D₂O exchangeable). IR (KBr) cm⁻¹: 3405, 3172, 2920, 1676. MS m/z: 368.1 (M⁺ + 1). |
| 6 | 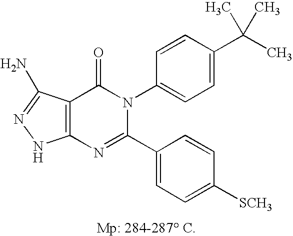 | Purity (HPLC): 99.8%, ¹H-NMR (CDCl₃): δ 1.28 (s, 9H), 2.41 (s, 3H), 5.3 (s, 2H, D₂O exchangeable), 7.03-7.05 (d, 2H), 7.12-7.14 (d, 2H), 7.21-7.23 (d, 2H), 7.30-7.32 (d, 2H), 12.45 (s, 1H, D₂O exchangeable). IR (KBr) cm ⁻¹: 3451, 3318, 3200, 2961, 1700. MS m/z: 406.4 (M⁺ + 1). |
| 7 | 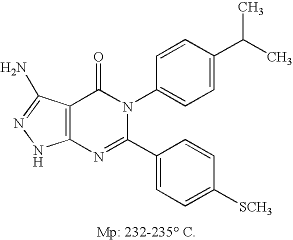 | Purity (HPLC): 99.83%, ¹H-NMR (CDCl₃): δ 1.20-1.22 (d, 6H), 2.17 (bs, 3H, D₂O exchangeable), 2.41 (s, 3H), 2.9-3.1 (m, 1H), 7.0-7.03 (d, 4H), 7.16-7.21 (m, 4H). IR (KBr) cm⁻¹: 3451, 3320, 3205, 2960, 1700. MS m/z: 392.1 (M⁺ + 1). |
| 8 | 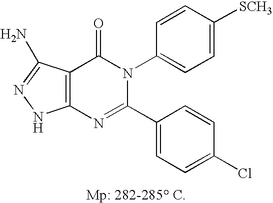 | Purity (HPLC): 99.89%, ¹H-NMR (DMSO-d₆): δ 2.08 (s, 3H), 5.34 (bs, 2H, D₂O exchangeable), 7.13-7.16 (m, 4H), 7.30-7.37 (m, 4H), 12.5 (1H, D₂O exchangeable). IR (KBr) cm⁻¹: 3495, 3405, 3303, 3174, 2920, 1671. MS m/z 384 (M⁺ + 1). |
| 9 | 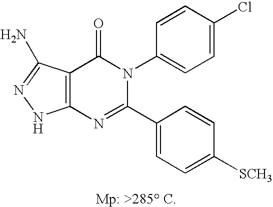 | Purity (HPLC): 99.88%, ¹H-NMR (DMSO-d₆): δ 2.43 (s, 3H), 5.34 (s, 2H, D₂O exchangeable), 7.08-7.10 (m, 2H), 7.24-7.26 (m, 2H), 7.31-7.38 (m, 4H), 12.48 (s, 1H, D₂O exchangeable). IR (KBr) cm⁻¹: 3448, 3092, 2920, 1694. MS m/z: 384 (M⁺ + 1). |
| 10 | 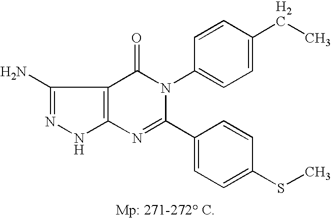 | Purity (HPLC): 91.83%, ¹H-NMR (DMSO-d₆): δ 1.1-1.17 (t, 3H), 2.40 (s, 3H), 2.49-2.58 (m, 2H), 5.30 (s, 2H, D₂O exchangeable), 7.04-7.06 (d, 2H), 7.12-7.24 (m, 4H), 7.35-7.38 (d, 2H), 12.44 (s, 1H, D₂O exchangeable). IR (KBr) cm⁻¹: 3377, 3294, 3182, 2964, 2917, 1698. MS m/z: 378.2 (M⁺ + 1). |
| 11 | 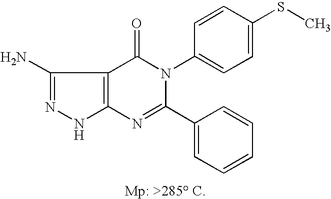 | Purity (HPLC): 99.51%, ¹H-NMR (DMSO-d₆): δ 2.41 (s, 3H), 5.32 (s, 2H, D₂O exchangeable), 7.10-7.15 (m, 4H), 7.18-7.24 (m, 3H), 7.32-7.34 (m, 2H), 12.46 (s, 1H, D₂O exchangeable). IR (KBr) cm⁻¹: 3462, 3315, 3197, 2916, 1693. MS m/z: 350.2 (M⁺ + 1). |
| 12 | 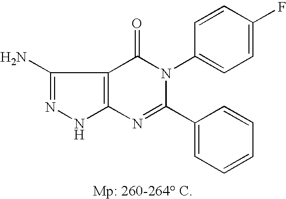 | Purity (HPLC): 98.98%, ¹H-NMR (DMSO-d₆): δ 5.34 (s, 2H, D₂O exchangeable), 7.11-7.13 (m, 2H), 7.22-7.30 (m, 7H), 12.49 (s, 1H, D₂O exchangeable). IR (KBr) cm⁻¹: 3455, 3144, 3090, 2921, 1694. MS m/z: 322.2 (M⁺ + 1). |
| 13 | 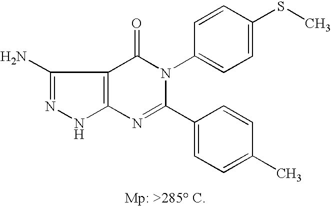 | Purity (HPLC): 99.87%, ¹H-NMR (DMSO-d₆): δ 2.22 (s, 3H), 2.42 (s, 3H), 5.31 (bs, 2H, D₂O exchangeable), 7.02-7.04 (d, 2H), 7.13-7.22 (m, 4H), 7.32-7.34 (m, 2H), 12.44 (s, 1H, D₂O exchangeable). IR (KBr) cm⁻¹: 3443, 3090, 2918, 1697. MS m/z: 364 (M⁺ + 1). |
| 14 | 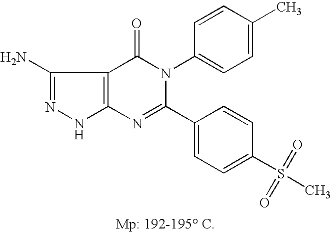 | Purity (HPLC): 97.98%, ¹H-NMR (DMSO-d₆): δ 2.23 (s, 3H), 3.23 (s, 3H), 5.31 (bs, 2H, D₂O exchangeable), 7.01-7.03 (d, 2H), 7.49-7.51 (d, 2H), 8.0-8.02 (d, 2H), 8.19-8.2 (d, 2H), 11.08 (bs, 1H, D₂O exchangeable), 12.5 (bs, 2H, D₂O exchangeable). IR (KBr) cm⁻¹: 3208, 2919, 1625, 1585, 1560. MS m/z: 396 (M⁺ + 1). |
| 15 | 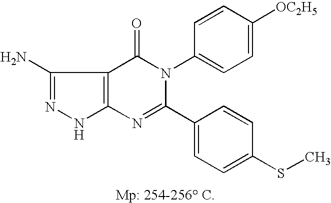 | Purity (HPLC): 99.77%, ¹H-NMR (DMSO-d₆): δ 1.27-1.3 (t, 3H), 2.41 (s, 3H), 3.93-3.98 (q, 2H), 5.56 (bs, 2H, D₂O exchangeable), 6.79-6.82 (d, 2H), 7.05-7.11 (m, 4H), 7.23-7.25 (d, 2H). IR (KBr) cm⁻¹: 3447, 3091, 2978, 2916, 1693. MS m/z: 394.1 (M⁺ + 1). |
| 16 | 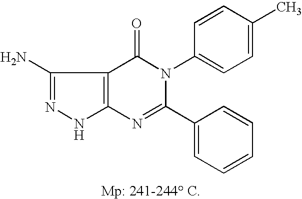 | Purity (HPLC): 99.17%, ¹H-NMR (DMSO-d₆): δ 2.22 (s, 3H), 5.31 (bs, 2H, D₂O exchangeable), 7.06 (bs, 4H), 7.19-7.25 (m, 3H), 7.30-7.31 (m, 2H), 12.45 (bs, 1H, D₂O exchangeable). IR (KBr) cm⁻¹: 3450, 3091, 2918, 1696. MS m/z: 318.2 (M⁺ + 1). |
| 17 | 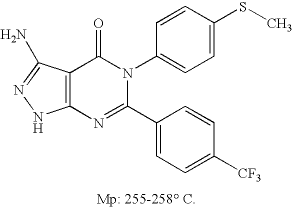 | Purity (HPLC): 99.68%, ¹H-NMR (DMSO-d₆): δ 2.41 (s, 3H), 5.35 (bs, 2H, D₂O exchangeable), 7.13-715 (m, 4H), 7.59-7.62 (m, 4H), 12.53 (bs, 1H, D₂O exchangeable). IR (KBr) cm⁻¹: 3333, 3217, 2923, 1694. MS m/z: 418.1 (M⁺ + 1). |
| 18 | 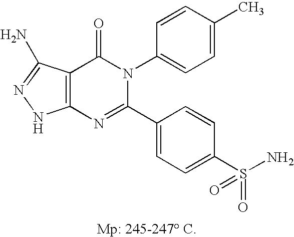 | Purity (HPLC): 98.6%, ¹H-NMR (DMSO-d₆): δ 2.5 (s, 3H), 7.19-7.20 (m, 1H), 7.35-7.44 (m, 4H, 1H D₂O exchangeable), 7.76-7.78 (m, 1H), 7.99-8.11 (m, 3H), 11.55 (s, 1H, D₂O exchangeable), 12.34 (s, 1H, D₂O exchangeable). IR (KBr) cm⁻¹: 3278, 2165, 1609, 1562. MS m/z: 397.1 (M⁺ + 1). |
| 19 | 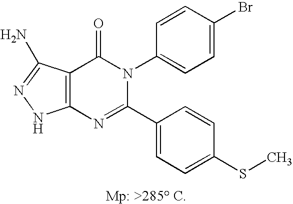 | Purity (HPLC): 97.6%, ¹H-NMR (DMSO-d₆): δ 2.42 (s, 3H), 5.33 (s, 2H, D₂O exchangeable), 7.08-7.1 (m, 2H), 7.24 (bs, 1H), 7.5-7.52 (m, 2H), 12.48 (s, 1H, D₂O exchangeable). IR (KBr) cm⁻¹: 3450, 3093, 2920, 1693. MS m/z: 429.9 (M⁺ + 1). |
| 20 | 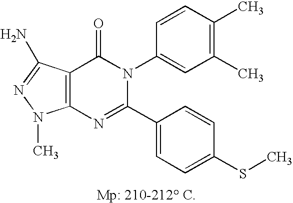 | Purity (HPLC): 99.7%, ¹H-NMR (CDCl₃): δ 2.18-2.21 (d, 6H), 2.43 (s, 3H), 3.81 (s, 3H), 4.5 (s, 2H, D₂O exchangeable), 7.76-6.78 (d, 1H), 6.89 (s, 1H), 7.03-7.05 (d, 3H), 7.23-7.26 (d, 2H). IR (KBr) cm⁻¹: 3414, 3309, 3217, 1700. MS m/z: 392.1 (M⁺ + 1). |
| 21 | 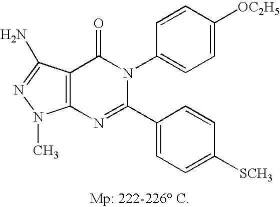 | Purity (HPLC): 95.4%, ¹H-NMR (CDCl₃): δ 1.38-1.41 (t, 3H), 2.43 (s, 3H), 3.38 (s, 3H), 3.96-4.02 (q, 2H), 4.55 (s, 2H, D₂O exchangeable), 6.79-6.80 (d, 2H), 6.97-6.99 (d, 2H), 7.04-7.06 (d, 2H), 7.21-7.23 (d, 2H). IR (KBr) cm⁻¹: 3416, 3307, 1701. MS m/z: 408.3 (M⁺ + 1). |
| 22 | 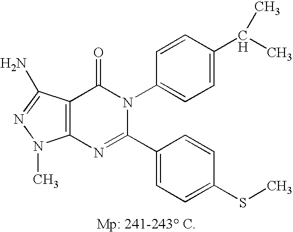 | Purity (HPLC): 99.5%, ¹H-NMR (DMSO-d₆): δ 1.13-1.15 (d, 6H), 2.4 (s, 3H), 2.81-2.88 (m, 1H), 3.67 (s, 3H), 5.42 (s, 2H, D₂O exchangeable), 7.05-7.07 (d, 2H), 7.11-7.18 (m, 4H), 7.23-7.25 (d, 2H). IR (KBr) cm⁻¹: 3408, 3300, 3184, 2959, 1699. MS m/z: 406.5 (M⁺ + 1). |
| 23 | 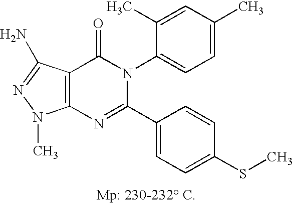 | Purity (HPLC): 99.8%, ¹H-NMR (DMSO-d₆): δ 1.96 (s, 3H), 2.22 (s, 3H), 2.41 (s, 3H), 3.67 (s, 3H), 5.44 (s, 2H, D₂O exchangeable), 6.93-6.95 (d, 1H), 7.0 (s, 1H), 7.07-7.1 (m, 3H), 7.24-7.26 (d, 2H). 7.05-7.07 (d, 2H), 7.11-7.18 (m, 4H), 7.23-7.25 (d, 2H). IR (KBr) cm⁻¹: 3410, 3303, 3186, 2922, 1701, 1636. MS m/z: 392.2 (M⁺ + 1). |
| 24 | 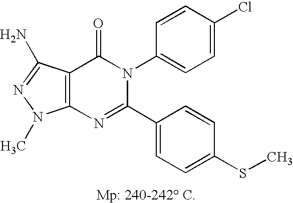 | Purity (HPLC): 98.5%, ¹H-NMR (CDCl₃): δ 2.44 (s, 3H), 3.8 (s, 3H), 4.5 (s, 2H, D₂O exchangeable), 7.03-7.07 (m, 4H), 7.19-7.21 (d, 2H), 7.28-7.30 (m, 2H). IR (KBr) cm⁻¹: 3416, 3313, 3218, 2920, 1701. MS m/z: 398.1 (M⁺ + 1). |
| 25 | 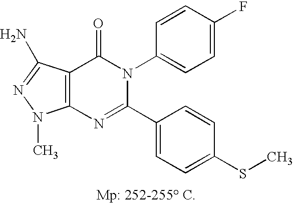 | Purity (HPLC): 97.9%, ¹H-NMR (DMSO-d₆): δ 2.42 (s, 3H), 3.6 (s, 3H), 5.45 (s, 2H, D₂O exchangeable), 7.08-7.13 (m, 2H), 7.15-7.17 (m, 2H), 7.25-7.33 (m, 4H). IR (KBr) cm⁻¹: 3414, 3304, 3184, 2921, 1699. MS m/z: 382.2 (M⁺ + 1). |
| 26 | 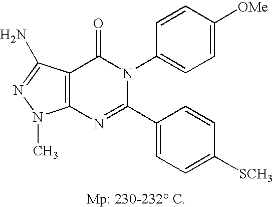 | Purity (HPLC): 96.8%, ¹H-NMR (CDCl₃): δ 2.43 (s, 3H), 3.78 (s, 3H), 3.82 (s, 3H), 4.56 (s, 2H, D₂O exchangeable), 6.81-6.83 (d, 2H), 6.99-7.01 (d, 2H), 7.04-7.06 (d, 2H), 7.21-7.23 (d, 2H). IR (KBr) cm⁻¹: 3397, 3299, 3182, 1698. MS m/z: 394.1 (M⁺ + 1). |
| 27 | 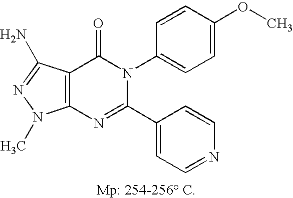 | Purity (HPLC): 99.4%, ¹H-NMR (CDCl₃): δ 3.68 (s, 3H0, 3.69 (s, 3H), 5.47 (s, 2H, D₂O exchangeable), 6.81-6.83 (q, 2H), 7.18-7.20 (d, 2H), 7.33-7.35 (d, 2H), 8.46-8.47 (d, 2H). IR (KBr) cm⁻¹: 3396, 3303, 3216, 1677. MS m/z: 349.2 (M⁺ + 1). |
| 28 | 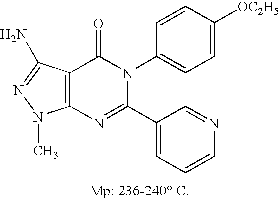 | Purity (HPLC): 99.76%, ¹H-NMR (DMSO-d₆): δ 1.26-1.29 (t, 3H), 2.5 (s, 3H), 3.68 (s, 3H), 3.92-3.97 (q, 2H), 5.45 (s, 2H, D₂O exchangeable), 6.80-6.82 (d, 2H), 7.16-7.18 (d, 2H), 7.27-7.29 (m, 1H), 7.72-7.74 (d, 1H), 8.42-8.44 (d, 1H), 8.53-8.54 (d, 1H). IR (KBr) cm⁻¹: 3422, 3362, 3211, 1709. MS m/z: 363.2 (M⁺ + 1). |
| 29 | 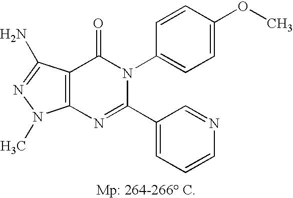 | Purity (HPLC): 99.5%, ¹H-NMR (DMSO-d₆): δ 2.5 (s, 3H), 3.68 (s, 3H), 3.69 (s, 3H), 5.45 (s, 2H, D₂O exchangeable), 6.82-6.84 (d, 2H), 7.18-7.20 (d, 2H), 7.27-7.29 (m, 1H), 7.72-7.74 (d, 1H), 8.42-8.44 (d, 1H), 8.53-8.54 (d, 1H). IR (KBr) cm⁻¹: 3414, 3339, 1693. MS m/z: 349.2 (M⁺ + 1). |
| 30 | 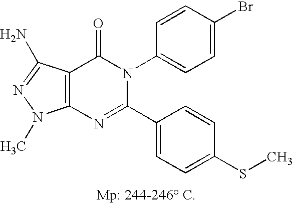 | Purity (HPLC): 99.5%, ¹H-NMR (DMSO-d₆): δ 2.50 (s, 3H), 3.68 (s, 3H), 5.44 (bs, 2H, D₂O exchangeable), 7.10-7.12 (d, 2H), 7.22-7.28 (m, 4H), 7.51-7.53 (d, 2H). IR (KBr) cm⁻¹: 3384, 2922, 1691.7. MS m/z: 443 (M⁺ + 1). |
| 31 | 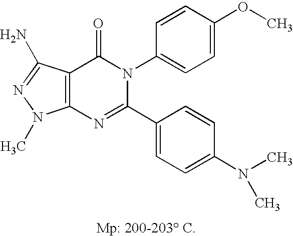 | Purity (HPLC): 94.6%, ¹H-NMR (CDCl₃): δ 2.93 (s, 6H), 3.79 (s, 3H), 3.82 (s, 3H), 4.54 (bs, 2H, D₂O exchangeable), (6.46-6.48 (d, 2H), 6.84-6.86 (m, 2H), 7.02-7.05 (m, 2H), 7.2-7.23 (m, 2H). IR (KBr) cm⁻¹: 3443, 3312.6, 3216.8, 2919, 2797, 1695. MS m/z: 391.1 (M⁺ + 1). |
| 32 | 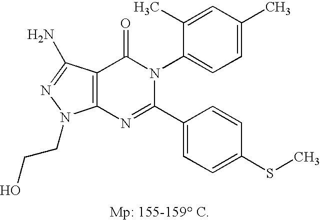 | Purity (HPLC): 96.8%, ¹H-NMR (CDCl₃): δ 2.04 (s, 3H), 2.29 (s, 3H), 2.43 (s, 3H), 3.76 (bs, 1H, D₂O exchangeable), 4.02 (m, 2H), 4.32-4.35 (m, 2H), 4.61 (bs, 2H, D₂O exchangeable), 6.9-7.04 (m, 5H), 7.2-7.22 (m, 2H). IR (KBr) cm⁻¹: 3394, 3223.7, 2924.4, 2881.9, 1705, 1678.2. MS m/z: 422.1 (M⁺ + 1). |
| 33 | 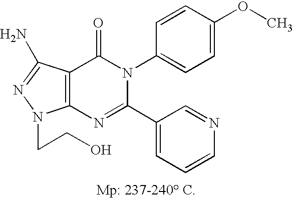 | Purity (HPLC): 95.1%, ¹H-NMR (DMSO-d₆): δ 3.7 (s, 3H), 3.73-3.77 (m, 2H), 4.09-4.12 (t, 2H), 4.83-4.86 (t, 2H), 5.47 (bs, 2H, D₂O exchangeable), 6.83-6.85 (m, 2H), 7.18-7.2 (m, 2H), 7.26-7.29 (m, 1H), 7.71-7.74 (m, 1H), 8.43-8.45 (m, 1H), 8.54-8.55 (m, 1H). IR (KBr) cm⁻¹: 3466.4, 3361.7, 3256, 2948.5, 2839.6, 1697.5. MS m/z: 379 (M⁺ + 1). |
| 34 | 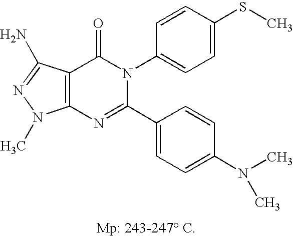 | Purity (HPLC): 96.9%, ¹H-NMR (CDCl₃): δ 2.47 (s, 3H), 2.94 (s, 6H), 3.82 (s, 3H), 4.53 (bs, 2H, D₂O exchangeable), 6.46-6.48 (d, 2H), 7.04-7.06 (d, 2H), 7.18-7.26 (m, 4H). IR (KBr) cm⁻¹: 3467.9, 3390, 3374, 2914, 2856, 1686 MS m/z: 407.1 (M⁺ + 1). |

### Example 35

### General Procedure

The compounds synthesized were further converted to the acyl derivatives using acetyl chloride or acetic anhydride in appropriate solvents according to the conventional procedures reported, to yield the title compounds. Purified by the recrystallization techniques.

### The Following Compounds Listed in Table-II are Prepared by the General Procedure Given in Example 35

TABLE II

| EXP. | STRUCTURE | ANALYTICAL DATA |
|---|---|---|
| 36 | 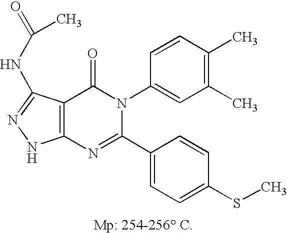 | Purity (HPLC): 96.7%, ¹H-NMR (DMSO-d₂): δ 2.08-2.15 (d, 6H), 2.40 (s, 3H), 2.61 (s, 3H), 6.89-6.91 (m, 1H), 7.02-7.07 (m, 4H), 7.26-7.28 (d, 2H), 7.77 (bs, 2H, D₂O exchangeable). IR (KBr) cm⁻¹: 3458, 3363, 2920, 1723, 1684. MS m/z: 420.1 (M⁺). |
| 37 | 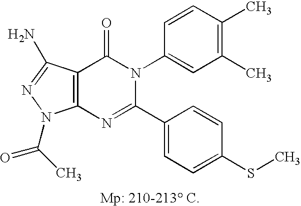 | Purity (HPLC): 95.99%, ¹H-NMR (DMSO-d₂): δ 2.14-2.17 (d, 6H), 2.5 (s, 3H), 2.62 (s, 3H), 6.02, (s, 2H, D₂O exchangeable), 6.96-6.98 (d, 1H), 7.07-7.09 (m, 4H), 7.3-7.32 (d, 2H). IR (KBr) cm⁻¹: 3481, 1702, 1623. MS m/z: 420.2 (M⁺ + 1). |
| 38 | 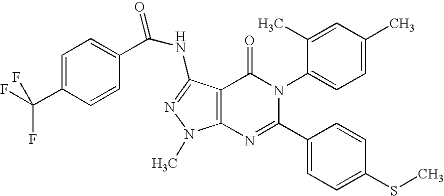 | Purity (HPLC): 94.7%, ¹H-NMR (CDCl₃): δ 2.03 (s, 3H), 2.31 (s, 3H), 2.45 (s, 3H), 4.07 (s, 3H), 6.98-7.07 (m, 4H), 7.21-7.30 (m, 3H), 7.69-7.71 (m, 2H), 8.07-8.09 (d, 2H), 9.30 (bs, 1H, D₂O exchangeable). MS m/z: 564.1 (M⁺ + 1). |
| 39 | 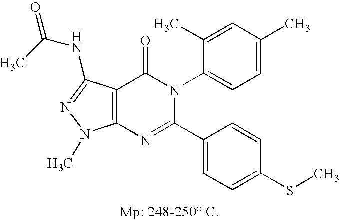 | Purity (HPLC): 100%, ¹H-NMR (CDCl₃): δ 2.02 (s, 3H), 2.30 (s, 3H), 2.44 (s, 3H), 4.00 (s, 3H), 6.91-7.06 (m, 5H), 7.25-7.26 (m, 2H), 8.37 (bs, 1H, D₂O exchangeable). IR (KBr) cm⁻¹: 3438, 2921.5, 1703, 1550.7. MS m/z: 434.1 (M⁺ + 1). |
| 40 | 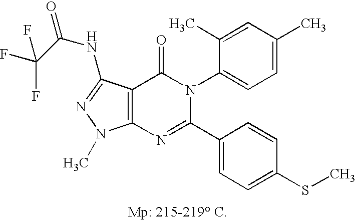 | Purity (HPLC): 94.3%, ¹H-NMR (CDCl₃): δ 2.01 (s, 3H), 2.29 (s, 3H), 2.44 (s, 3H), 4.05 (s, 3H), 6.93-6.95 (m, 1H), 7.00-7.07 (m, 4H), 7.28 (m, 1H), 9.11 (bs, 1H, D₂O exchangeable) IR (KBr) cm⁻¹: 3432, 2924, 2854, 2364, 1752, 1699.8. MS m/z: 488.1 (M⁺ + 1). |
| 41 | 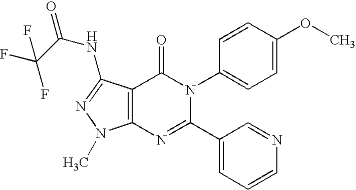 | Purity (HPLC): 99.7%, ¹H-NMR (CDCl₃): δ 3.79 (s, 3H), 4.06 (s, 3H), 6.86-6.88 (m, 2H), 7.01-7.03 (m, 2H), 7.2-7.22 (m, 1H), 7.6-7.62 (m, 1H), 8.54-8.56 (m, 1H), 8.64-8.65 (d, 1H), 9.1 (bs, 1H, D₂O exchangeable). MS m/z: 445.1 (M⁺ + 1). |
| 42 | 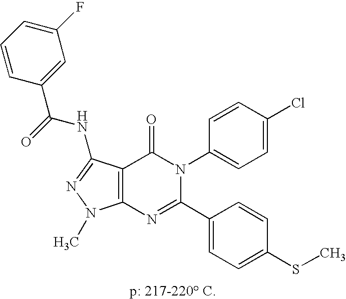 | Purity (HPLC): 97.1%, ¹H-NMR (CDCl₃): δ 2.46 (s, 3H), 4.06 (s, 3H), 7.07-7.10 (m, 4H), 7.24-7.36 (m, 6H), 7.69-7.71 (d, 2H), 9.15 (bs, 2H, D₂O exchangeable). IR (KBr) cm⁻¹: 3402, 3277, 3066, 2922, 1698.2. MS m/z: 520.1 (M⁺ + 1). |

Described below are the examples of pharmacological assays used for finding out the efficacy of the compounds of the present invention wherein their protocols and results are provided.

### In Vitro Evaluation of Cyclooxygenase-2 (COX-2) Inhibition Activity

The compounds of this invention exhibited in vitro inhibition of COX-2. The COX-2 inhibition activities of the compounds illustrated in the examples were determined by the following method.

### Human Whole Blood Assay:

Human whole blood provides a protein and cell rich milieu appropriate for the study of biochemical efficacy of anti-inflammatory compounds such as selective COX-2 inhibitors. Studies have shown that normal human blood does not contain COX-2 enzyme. This is correlating with the observation that COX-2 inhibitors have no effect on prostaglandin E₂ (PGE₂) production in normal blood. These inhibitors were active only after incubation of human blood with lipopolysaccharide (LPS), which induces COX-2 production in the blood.

Fresh blood was collected in tubes containing sodium heparin by vein puncture from healthy male volunteers. The subjects should have no apparent inflammatory conditions and not taken NSAIDs for at least 7 days prior to blood collection. Blood was preincubated with aspirin in vitro (12 μg/ml, at time zero) to inactivate COX-1 for 6 hours. Then test compounds (at various concentrations) or vehicle were added to blood. After that blood was stimulated with LPS B:4 (10 μg/ml) and incubated for another 18 h at 37° C. water bath. After which the blood was centrifuged, plasma was separated and stored at −80° C. (J. Pharmacol. Exp. Ther, 271, 1705, 1994; Proc. Natl. Acad. Sci. USA, 96, 7563, 1999). The plasma was assayed for PGE2 using Cayman ELISA kit as per the procedure outlined by the manufacturer (Cayman Chemicals, Ann Arbor, USA). Representative results of PGE2 inhibition are shown in Table I.

TABLE I

% PGE2 Inhibition

| Example No | 0.25 μM | 10 μM |
|---|---|---|
| 1 | 20.33 | 41.58 |
| 2 | 21.92 | 16.83 |
| 5 | — | 31.81 |
| 6 | 18.59 | 37.69 |
| 8 | 28.27 | — |
| 9 | — | 27.13 |
| 11 | — | 29.19 |
| 12 | 26.2 | — |
| 13 | 26.84 | — |
| 14 | 19.28 | 23 |
| 15 | 20.22 | — |
| 16 | 31.4 | — |
| 17 | 19.36 | 36.51 |

### COX-1 and COX-2 Enzyme Based Assay

COX-1 and COX-2 enzyme based assays were carried out to check the inhibitory potential of test compounds on the production of prostaglandin by purified recombinant COX-1/COX-2 enzyme (Proc. Nat. Acad. Sci. USA, 88, 2692-2696, 1991; J. Clin. Immunoassay 15, 116-120, 1992) In this assay, the potential of test compound to inhibit the production of prostaglandin either by COX-1 or COX-2 from arachidonic acid (substrate) was measured. This was an enzyme based in-vitro assay to evaluate selective COX inhibition with good reproducibility.

Arachidonic acid was converted to PGH2 (Intermediate product) by COX1/COX-2 in presence or absence of the test compound. The reaction was carried out at 37° C. and after 2 minutes it was stopped by adding 1M HCl. Intermediate product PGH2 was converted to a stable prostanoid product PGF2α by SnCl2 reduction. The amount of PGF2α produced in the reaction was inversely proportional to the COX inhibitory potential of the test compound. The prostanoid product was quantified via enzyme immunoassay (EIA) using a broadly specific antibody that binds to all the major forms of prostaglandin, using Cayman ELISA kit as per the procedure outlined by the manufacturer (Cayman Chemicals, Ann Arbor, USA). Representative results of inhibition are shown in Table II.

TABLE II

| Example | Conc. | COX-1 | COX-2 |
|---|---|---|---|
| No. | (μM) | Inhibition (%) | Inhibition (%) |
| 23 | 1 | 51.62 | Not Active |
| 40 | 10 | 3.11 | 15.54 |

### In Vitro Measurement of Tumor Necrosis Factor Alpha (TNF-α)

This assay determines the effect of test compounds on the production of TNF α in human Peripheral Blood Mononuclear Cells (PBMC). Compounds were tested for their ability to inhibit the activity of TNF α in human PBMC. PBMC were isolated from blood (from healthy volunteers) using BD Vacutainer CPT™ (Cell preparation tube, BD Bio Science) and suspended in RPMI medium (Physiol. Res. 52: 593-598, 2003). The test compounds were pre-incubated with PBMC (0.5 million/incubation well) for 15 minutes at 37° C. and then stimulated with Lipopolysaccharide (Escherichia coli: B4; 1 μg/ml) for 18 h at 37° C. in 5% CO₂. The levels of TNFα in cell culture medium were estimated using enzyme linked Immunosorbent assay performed in a 96 well format as per the procedure of the manufacturer (Cayman Chemical, Ann Arbor, USA). Representative results of TNF-α inhibition are shown in Table III.

TABLE III

| | % TNF-α |
|---|---|
| Example | Inhibition |
| No | 10 μM |
| 2 | 53.42 |
| 19 | 64.79 |
| 21 | 75.13 |
| 22 | 53.97 |
| 23 | 86.34 |
| 24 | 87.88 |
| 25 | 52.85 |
| 30 | 86.57 |
| 31 | 87.86 |
| 36 | 29.91 |
| 37 | 29.85 |
| 40 | 78.04 |

### In Vitro Measurement of Interleukin-6 (IL-6)

This assay determines the effect of test compounds on the production of IL-6 in human PBMC (Physiol. Res. 52: 593-598, 2003). Compounds were tested for their ability to inhibit the activity of IL-6 in human PBMC. PBMC were isolated from blood using BD Vacutainer CPT™ Cell preparation tube (BD Bio Science) and suspended in RPMI medium. The test compounds were pre-incubated with PBMC (0.5 million/incubation well) for 15 minutes at 37° C. and then stimulated with Lipopolysaccharide (Escherichia coli: B4; 1 μg/ml) for 18 h at 37° C. in 5% CO₂. The levels of IL-6 in cell culture medium were estimated using enzyme linked Immunosorbent assay performed in a 96 well format as per the procedure of the manufacturer (Cayman Chemical, Ann Arbor, USA). Representative results of IL-6 inhibition are shown in Table IV.

TABLE IV

| Example | % IL-6 Inhibition |
|---|---|
| No | (1 μM) |
| 19 | 15.93 |
| 22 | 12 |
| 36 | 12.67 |

### Carrageenan Induced Paw Edema Test in Rat

The carrageenan paw edema test was performed as described by Winter et al (Proc. Soc. Exp. Biol. Med, 111, 544, 1962). Male wistar rats were selected with body weights equivalent within each group. The rats were fasted for eighteen hours with free access to water. The rats were dosed orally with the test compound suspended in vehicle containing 0.25% carboxymethylcellulose and 0.5% Tween 80. The control rats were administered with vehicle alone. After an hour, the rats were injected with 0.1 ml of 1% Carrageenan solution in 0.9% saline into the sub-plantar surface of the right hind paw. Paw volume was measured using digital plethysmograph before and after 3 hours of carrageenan injection. The average of foot swelling in drug treated animals was compared with that of control animals. Anti-inflammatory activity was expressed as the percentage inhibition of edema compared with control group [Arzneim-Forsch/Drug Res., 43 (1), 1, 44-50, 1993; Ottemess and Bliven, Laboratory Models for Testing NSAIDs, In Non-Steroidal Anti-Inflammatory Drugs, (J. Lombardino, ed. 1985)]. Representative results of edema inhibition are shown in Table V.

### Ulcerogenic Potential

In order to evaluate compound's role on the ulcer formation, the animals were sacrificed and the stomach was taken out and flushed with 1% formalin. Animals (male wistar 200 gm) were fasted for 18 hrs free access to water and the test compounds were suspended in 0.5% Tween 80 and 0.25% CMC (carboxymethylcellulose) solution to make a uniform suspension. After 4 hrs of oral administration of test compounds, all the animals were sacrificed by cervical dislocation. Dissect the stomach carefully and filled up with a sterile saline solution and embedded in 6% formalin solution. Finally cut the stomach longitudinally and ulcer lesions were observed with computerized stereomicroscope. Compare the test compound treated groups with the vehicle treated groups. Dose selected: 50, 100, 200 mg/kg (Marco Romano et al, Journal of clinical Investigation, 1992; 2409-2421). Representative results of ulcer incidence are shown in Table V.

TABLE V

| | | Ulcer Incidence |
|---|---|---|
| | Rat Paw Edema model | (Number of |
| | % Inhibition | animals/Total number |
| Example No | (5 mg/kg b. wt., p.o.) | of animals) |
| 2 | 30.52 + 10.3 | No ulcer |

### Inhibitory Action on Adjuvant Arthritis

Compounds were assayed for their activity on rat adjuvant induced arthritis according to Theisen-Popp et al., (Agents Actions, 42, 50-55, 1994). Six to seven weeks old, Wistar rats were weighed, marked and assigned to groups [a negative control group in which arthritis was not induced (non-adjuvant control), a vehicle-treated arthritis control group, test substance treated arthritis group]. Adjuvant induced arthritis was induced by an injection of Mycobacteriuni butyricun (Difco) suspended in liquid paraffin into the sub-plantar region of the right hind paw (J. Pharmacol. Exp. Ther., 284, 714, 1998). Body weight, contra-lateral paw volumes were determined at various days (0, 4, 14, 21) for all the groups. The test compound or vehicle was administered orally beginning post injection of adjuvant and continued for 21 days. On day 21, body weight and paw volume of both right and left hind paw, spleen, and thymus weights were determined. In addition, the radiograph of both hind paws was taken to assess the tibio-tarsal joint integrity. Hind limb below the stifle joint was removed and fixed in 1% formalin saline. At the end of the experiment, plasma samples were analysed for cytokines, interleukin and prostaglandin. The presence or absence of lesions in the stomachs was also observed.

Two-factor (‘treatment’ and ‘time’) Analysis of Variance with repeated measures on ‘time’ were applied to the % changes for body weight and foot volumes. A post hoc Dunnett's test was conducted to compare the effect of treatments to vehicle. A one-way Analysis of Variance was applied to the thymus and spleen weights followed by the Dunnett's test to compare the effect of treatments to vehicle. Dose-response curves for % inhibition in foot volumes on days 4, 14 and 21 were fitted by a 4-parameter logistic function using a nonlinear Least Squares' regression. ID₅₀ was defined as the dose corresponding to a 50% reduction from the vehicle and was derived by interpolation from the fitted 4-parameter equation.

### LPS Induced Sepsis for Measurement of TNF-α Inhibition in Mice

The LPS induced sepsis model in mice was performed as described by Les sekut et al (J Lab Clin Med 1994; 124:813-20). Female Swiss albino mice were selected and the body weights were equivalent within each group. The mice were fasted for 20 hours with free access to water. The mice were dosed orally with the test compound suspended in vehicle containing 0.5% Tween 80 in 0.25% Carboxy-methylcellulose sodium salt. The control mice were administered the vehicle alone. After 30 min of oral dosing, mice were injected with 500 μg of Lipopolysaccharide (Escherichia coli, LPS: B4 from Sigma) in phosphate buffer saline solution into the intraperitoneal cavity of the mice. After 90 min of LPS administration mice were bled via retro-orbital sinus puncture. Blood samples were stored overnight at 4° C. Serum samples were collected by centrifuging the samples at 400 rpm for 15 min at 4° C. Immediately the serum samples were analysed for TNFα levels using commercially available mouse TNF-α ELISA kit (Amersham Biosciences) and assay was performed by the manufacturer instruction. Representative results of TNF-α inhibition are shown in Table VI.

TABLE VI

| Example No. | TNF-α Inhibition (%) |
|---|---|
| 2 | 34.29 (at 50 mg/kg) |
| 21 | 75.15 (at 50 mg/kg) |
| 23 | 92.02 (at 5 mg/kg) |
| 24 | 91.99 (at 50 mg/kg) |

DTP Human Tumor Cell Line Screen

### Methodology of the In Vitro Cancer Screen

The three cell line, one-dose prescreen carried out which identifies a large proportion of the compounds that would be inactive in multi-dose 60 cell line screening. The current assay utilizes a 384 well plate format and fluorescent staining technologies resulting in greater screening capacity for testing of synthetic samples.

### Cell Lines

The cell lines of the cancer-screening panel are grown in RPMI 1640 medium containing 5% fetal bovine serum and 2 mM L-glutamine. For a typical screening experiment, cells are inoculated into 96 well microtiter plates in 100 μL. After cell inoculation, the micro-titer plates are incubated at 37° C., 5% CO₂, 95% air and 100% relative humidity for 24 h prior to addition of experimental drugs. The cells are plated a densities of 5000 cells/well (MCF7), 1000 cells/well (NCI-H460), and 7500 cells/well (SF-268) to allow for varying doubling time of the cell lines. Each plate contains all three-cell lines, a series of dilutions of standard agents, total kill wells and appropriate controls. Plates are incubated under standard conditions for 24 hours prior to addition of experimental compounds or extracts.

### Addition of Experimental Agents (Pure Compounds)

Experimental compounds are solubilized in dimethyl sulfoxide (DMSO) at 400-times the desired maximum test concentration (maximum final DMSO concentration of 0.25%) and stored frozen. Compounds are then diluted with complete media with 0.1% gentamicin sulfate (5 μl of test sample in 100% DMSO is added to 565 μl of complete medium). 20 μl of this solution is then dispensed into test wells containing 50 μl of cell suspension to yield a test concentration of 1.00E-04M.

Two standard drugs, meaning that their activities against the cell lines are well documented, are tested against each cell line: NSC 19893 (5-FU) and NSC 123127 (Adriamycin).

### Endpoint Measurement

After compound addition, plates are incubated at standard conditions for 48 hours, 10 μl/well Alamar Blue is added and the plates are incubated for an additional 4 hours. Fluorescence is measured using an excitation wavelength of 530 nm and an emission wavelength of 590 nm.

Calculation of Percent Test Cell Growth/Control (untreated) Cell Growth (T/C)
Calculation of Percent Test Cell Growth/Control (untreated) Cell Growth (T/C)

Percent growth is calculated on a plate-by-plate basis for test wells relative to control wells. Percent Growth is expressed as the ratio of fluorescence of the test well to the average fluorescence of the control wells ×100. Representative results of T/C are shown in Table VII.

TABLE VII

| | % T/C (100 μM) | | |
|---|---|---|---|
| Example | Lung | Breast | CNS |
| No. | (NCI-H460) | (MCF-7) | (SF-268) |
| 2 | 7 | 31 | 39 |
| 6 | 0 | 0 | 1 |
| 8 | 2 | 26 | 36 |
| 9 | 4 | 9 | 31 |
| 10 | 4 | 22 | 10 |
| 17 | 25 | 0 | 0 |

1. Novel pyrazolopyrimidinones of formula (I) 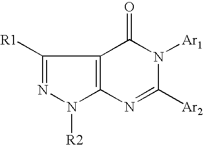
their derivatives, their pharmaceutically acceptable salts and their pharmaceutically acceptable compositions, wherein Ar₁ and Ar₂ may be same or different and independently represent substituted or unsubstituted groups selected from the group consisting of aryl, heteroaryl, and heterocyclyl group; R₁ represents amino, hydrazine, alkylamino, arylamino, acylamino, sulfonylamino, substituted (C₁-C₆)alkyl, —NHCH₂CN, —NHCH₂C(═NH)NHOH, —NHCONH₂, —NHCSNH₂, —NHCONH-alkyl, —NHCONH-aryl, —NHCSNH-alkyl, —NHCSNH-aryl, —NHCO-piperzine, —NHCS-piperzine, —NHCO-aryl, or —NHCO-heteroaryl; R₂ represents hydrogen, hydroxy, nitro, nitroso, alkyl, azido, —C(═NH)NH₂, halogen, formyl or substituted or unsubstituted groups selected from the group consisting of haloalkyl, alkoxy, aryloxy, aralkyl, aralkoxy, heteroaryl, heterocyclyl, acyl, acyloxy, cycloalkyl, amino, monoalkylamino, dialkylamino, acylamino, alkylsulfonyl, arylsulfonyl, alkylsulfinyl, arylsulfinyl, alkylthio, arylthio, alkoxycarbonyl, aryloxycarbonyl, alkoxyalkyl, sulfamoyl, and carboxylic acid and its derivatives.

2. Novel pyrazolopyrimidinones as claimed in claim 1, wherein groups represented by Ar₁ and Ar₂ are substituted or unsubstituted and are selected from the group consisting of phenyl, naphthyl, pyridyl, thienyl, furyl, pyrrolyl, oxazolyl, thiazolyl, imidazolyl, oxadiazolyl, thiadiazolyl, tetrazolyl, pyrimidinyl, pyrazine, piperazine, benzopyranyl, benzofuranyl, benzimidazolyl, benzoxazolyl, benzothiazolyl, benzopyrrolyl, benzoxadiazolyl, benzothiadiazolyl, pyrrolidinyl, thiazolidinyl, oxazolidinyl, morpholinyl, thiomorpholinyl, piperidinyl and piperazinyl.

3. Novel pyrazolopyrimidinones as claimed in claim 1, selected from the group consisting of:
1) 3-Amino-5-(4-methylphenyl)-6-[4-(methylthio)phenyl]-1,5-dihydro-4H-pyrazolo[3,4-d]pyrimidin-4-one;
2) 3-Amino-5-(3,4-dimethylphenyl)-6-[4-(methylthio)phenyl]-1,5-dihydro-4H-pyrazolo[3,4-d]pyrimidin-4-one;
3) 3-Amino-5-(4-fluorophenyl)-6-[4-(methylthio)phenyl]-1,5-dihydro-4H-pyrazolo[3,4-d]pyrimidin-4-one;
4) 3-Amino-5-[(4-methylthio)phenyl]-6-(4-fluorophenyl)-1,5-dihydro-4H-pyrazolo[3,4-d]pyrimidin-4-one;
5) 3-Amino-5-(4-tert-butylphenyl)-6-[4-(methylthio)phenyl]-1,5-dihydro-4H-pyrazolo[3,4-d]pyrimidin-4-one;
6) 3-Amino-5-(4-isopropylphenyl)-6-[(4-methylthio)phenyl]-1,5-dihydro-4H-pyrazolo[3,4-d]pyrimidin-4-one;
7) 3-Amino-5-[(4-methylthio)phenyl]-6-(4-chlorophenyl)-1,5-dihydro-4H-pyrazolo[3,4-d]pyrimidin-4-one;
8) 3-Amino-5-(4-chlorophenyl)-6-[(4-methylthio)phenyl]-1,5-dihydro-4H-pyrazolo[3,4-d]pyrimidin-4-one;
9) 3-Amino-5-(4-ethylphenyl)-6-[(4-methylthio)phenyl]-1,5-dihydro-4H-pyrazolo[3,4-d]pyrimidin-4-one;
10) 3-Amino-5-[(4-methylthio)phenyl]-6-phenyl-1,5-dihydro-4H-pyrazolo[3,4-d]pyrimidin-4-one;
11) 3-Amino-5-(4-fluorophenyl)-6-phenyl-1,5-dihydro-4H-pyrazolo[3,4-d]pyrimidin-4-one;
12) 3-Amino-5-[(4-methylthio)phenyl]-6-(4-methylphenyl)-1,5-dihydro-4H-pyrazolo[3,4-d]pyrimidin-4-one;
13) 3-Amino-5-(4-methylphenyl)-6-[(4-methylsulfonyl)phenyl]-1,5-dihydro-4H-pyrazolo[3,4-d]pyrimidin-4-one;
14) 3-Amino-5-(4-ethoxyphenyl)-6-[(4-methylthio)phenyl]-1,5-dihydro-4H-pyrazolo[3,4-d]pyrimidin-4-one;
15) 3-Amino-5-(4-methylphenyl)-6-phenyl-1,5-dihydro-4H-pyrazolo[3,4-d]pyrimidin-4-one;
16) 3-Amino-5-[(4-methylthio)phenyl]-6-(4-trifluoromethylphenyl)-1,5-dihydro-4H-pyrazolo[3,4-d]pyrimidin-4-one;
17) 4-[3-Amino-5-(4-methylphenyl)-4-oxo-4,5-dihydro-1H-pyrazolo[3,4-d]pyrimidin-6-yl]benzenesulfonamide;
18) 3-Amino-5-(4-bromophenyl)-6-[(4-methylthio)phenyl]-1,5-dihydro-4H-pyrazolo[3,4-d]pyrimidin-4-one;
19) 3-Amino-5-(3,4-dimethylphenyl)-1-methyl-6-[4-(methylthio)phenyl]-1,5-dihydro-4H-pyrazolo[3,4-d]pyrimidin-4-one;
20) 3-Amino-5-(4-ethoxyphenyl)-1-methyl-6-[4-(methylthio)phenyl]-1,5-dihydro-4H-pyrazolo[3,4-d]pyrimidin-4-one;
21) 3-Amino-5-(4-isopropylphenyl)-1-methyl-6-[4-(methylthio)phenyl]-1,5-dihydro-4H-pyrazolo[3,4-d]pyrimidin-4-one;
22) 3-Amino-5-(2,4-dimethylphenyl)-1-methyl-6-[4-(methylthio)phenyl]-1,5-dihydro-4H-pyrazolo[3,4-d]pyrimidin-4-one;
23) 3-Amino-5-(4-chlorophenyl)-1-methyl-6-[4-(methylthio)phenyl]-1,5-dihydro-4H-pyrazolo[3,4-d]pyrimidin-4-one;
24) 3-Amino-5-(4-fluorophenyl)-1-methyl-6-[4-(methylthio)phenyl]-1,5-dihydro-4H-pyrazolo[3,4-d]pyrimidin-4-one;
25) 3-Amino-5-(4-methoxyphenyl)-1-methyl-6-[4-(methylthio)phenyl]-1,5-dihydro-4H-pyrazolo[3,4-d]pyrimidin-4-one;
26) 3-Amino-5-(4-methoxyphenyl)-1-methyl-6-pyridin-4-yl-1,5-dihydro-4H-pyrazolo[3,4-d]pyrimidin-4-one;
27) 3-Amino-5-(4-ethoxyphenyl)-1-methyl-6-pyridin-3-yl-1,5-dihydro-4H-pyrazolo[3,4-d]pyrimidin-4-one;
28) 3-Amino-5-(4-methoxyphenyl)-1-methyl-6-pyridin-3-yl-1,5-dihydro-4H-pyrazolo[3,4-d]pyrimidin-4-one;
29) 3-Amino-5-(4-bromophenyl)-1-methyl-6-[4-(methylthio)phenyl]-1,5-dihydro-4H-pyrazolo[3,4-d]pyrimidin-4-one;
30) 3-Amino-6-[4-(dimethylamino)phenyl]-5-(4-methoxyphenyl)-1-methyl-1,5-dihydro-4H-pyrazolo[3,4-d]pyrimidin-4-one;
31) 3-Amino-5-(2,4-dimethylphenyl)-1-(2-hydroxyethyl)-6-[4-(methylthio)phenyl]-1,5-dihydro-4H-pyrazolo[3,4-d]pyrimidin-4-one;
32) 3-Amino-1-(2-hydroxyethyl)-5-(4-methoxyphenyl)-6-pyridin-3-yl-1,5-dihydro-4H-pyrazolo[3,4-d]pyrimidin-4-one;
33) 3-Amino-6-[4-(dimethylamino)phenyl]-1-methyl-5-[(4-methylthio)phenyl]-1,5-dihydro-4H-pyrazolo[3,4-d]pyrimidin-4-one;
34) N-[5-(3,4-Dimethylphenyl)-4-oxo-6-[(4-methylthio)phenyl]-4,5-dihydro-1H-pyrazolo[3,4-d]pyrimidin-3-yl]acetamide;
35) 1-Acetyl-3-amino-5-(3,4-dimethylphenyl)-6-[(4-methylthio)phenyl]-1,5-dihydro-4H-pyrazolo[3,4-d]pyrimidin-4-one;
36) 3-Amino-5-(4-methoxyphenyl)-6-[4-(methylthio)phenyl]-1,5-dihydro-4H-pyrazolo[3,4-d]pyrimidin-4-one;
37) N-(4-Oxo-5-(2,4-dimethylphenyl)-1-methyl-6-[(4-methylthio)phenyl]-4,5-dihydro-1H-pyrazolo[3,4-d]pyrimidin-3-yl)-4-(trifluoromethyl)benzamide;
38) N-{5-(2,4-Dimethylphenyl)-1-methyl-6-[4-(methylthio)phenyl]-4-oxo-4,5-dihydro-1H-pyrazolo[3,4-d]pyrimidin-3-yl}acetamide;
39) 2,2,2-Trifluoro-N-{5-(2,4-dimethylphenyl)-1-methyl-6-[4-(methylthio)phenyl]-4-oxo-4,5-dihydro-1H-pyrazolo[3,4-d]pyrimidin-3-yl}acetamide;
40) 2,2,2-Trifluoro-N-[5-(4-methoxyphenyl)-1-methyl-4-oxo-6-pyridin-3-yl-4,5-dihydro-1H-pyrazolo[3,4-d]pyrimidin-3-yl]acetamide;
41) N-(4-Oxo-5-(4-chlorophenyl)-1-methyl-6-[(4-methylthio)phenyl]-4,5-dihydro-1H-pyrazolo[3,4-d]pyrimidin-3-yl)-3-fluorobenzamide;
42) 3-Amino-5-(4-methoxyphenyl)-6-[4-methylphenyl]-1,5-dihydro-4H-pyrazolo[3,4-d]pyrimidin-4-one;
43) 3-Amino-5-(4-methoxyphenyl)-6-[4-methoxyphenyl]-1,5-dihydro-4H-pyrazolo[3,4-d]pyrimidin-4-one;
44) 3-Amino-5-(4-methoxyphenyl)-6-[4-chlorophenyl]-1,5-dihydro-4H-pyrazolo[3,4-d]pyrimidin-4-one;
45) 3-Amino-5-(4-chlorophenyl)-6-[4-chlorophenyl]-1,5-dihydro-4H-pyrazolo[3,4-d]pyrimidin-4-one;
46) 3-Amino-5-(4-chlorophenyl)-6-[4-methoxyphenyl]-1,5-dihydro-4H-pyrazolo[3,4-d]pyrimidin-4-one;
47) 3-Amino-5-(4-chlorophenyl)-6-[4-methylphenyl]-1,5-dihydro-4H-pyrazolo[3,4-d]pyrimidin-4-one;
48) 3-Amino-5-(4-fluorophenyl)-6-[4-chlorophenyl]-1,5-dihydro-4H-pyrazolo[3,4-d]pyrimidin-4-one;
49) 3-Amino-5-(4-fluorophenyl)-6-[4-methoxyphenyl]-1,5-dihydro-4H-pyrazolo[3,4-d]pyrimidin-4-one;
50) 3-Amino-5-(4-fluorophenyl)-6-[4-methylphenyl]-1,5-dihydro-4H-pyrazolo[3,4-d]pyrimidin-4-one;
51) 3-Amino-5-(4-trifluoromethylphenyl)-6-[4-chlorophenyl]-1,5-dihydro-4H-pyrazolo[3,4-d]pyrimidin-4-one;
52) 3-Amino-5-(4-trifluoromethylphenyl)-6-[4-methoxyphenyl]-1,5-dihydro-4H-pyrazolo[3,4-d]pyrimidin-4-one;
53) 3-Amino-5-(4-trifluoromethylphenyl)-6-[4-methylphenyl]-1,5-dihydro-4H-pyrazolo[3,4-d]pyrimidin-4-one;
54) 3-Amino-5-(4-trifluoromethylphenyl)-6-[4-(methylthio)phenyl]-1,5-dihydro-4H-pyrazolo[3,4-d]pyrimidin-4-one;
55) 3-Amino-5-(4-methylsulphonyl)-6-[4-chlorophenyl]-1,5-dihydro-4H-pyrazolo[3,4-d]pyrimidin-4-one;
56) 3-Amino-5-(4-methylsulphonyl)-6-[4-methoxyphenyl]-1,5-dihydro-4H-pyrazolo[3,4-d]pyrimidin-4-one;
57) 3-Amino-5-(4-methylsulphonyl)-6-[4-methylphenyl]-1,5-dihydro-4H-pyrazolo[3,4-d]pyrimidin-4-one;
58) 3-Amino-5-(4-methylsulphonyl)-6-[4-(methylthio)phenyl]-1,5-dihydro-4H-pyrazolo[3,4-d]pyrimidin-4-one;
59) 3-Amino-5-[4-(methylthio)phenyl]-6-pyridin-4-yl-1,5-dihydro-4H-pyrazolo[3,4-d]pyrimidin-4-one;
60) 3-Amino-5-(4-ethoxyphenyl)-1-methyl-6-pyridin-4-yl-1,5-dihydro-4H-pyrazolo[3,4-d]pyrimidin-4-one;
61) 1-{1-Methyl-5-[4-(methylthio)phenyl]-4-oxo-6-phenyl-4,5-dihydro-1H-pyrazolo[3,4-d]pyrimidin-3-yl}urea; and
62) 1-{5-[4-(Methylthio)phenyl]-4-oxo-6-phenyl-4,5-dihydro-1H-pyrazolo[3,4-d]pyrimidin-3-yl}urea.

4. A pharmaceutical composition, which comprises pyrazolopyrimidinones as defined in claim 1 and a pharmaceutically acceptable carrier, diluent, excipient or solvate.

5. A pharmaceutical composition as claimed in claim 4, in the form of a tablet, capsule, powder, syrup, solution, aerosol or suspension.

6. A method for the prophylaxis or treatment of inflammation, rheumatoid arthritis, osteoporosis, uveititis, acute and chronic myelogenous leukemia, atherosclerosis, cancer, pancreatic β cell destruction, osteoarthritis, rheumatoid spondylitis, gouty arthritis, inflammatory bowel disease, psoriasis, adult respiratory distress syndrome (ARDS) and asthma which comprises administering a therapeutically effective amount of the pyrazolopyrimidinone of claim 1.

7. A method according to claim 6 wherein the therapeutically effective amount inhibits the production of cytokines as selected from TNF-α, and IL-6.
